(19) United States
(12) Patent Application Publication
(10) Pub. No.: US 2021/0139999 A1
(43) Pub. Date: May 13, 2021

(54) PROGNOSTIC AND TREATMENT RESPONSE PREDICTIVE METHOD

(71) Applicant: Cancer Research Technology Limited, London (GB)

(72) Inventors: Christian P. Bromley, London (GB); Eduado Bonavita, London (GB); Santiago P. Zelenay, London (GB)

(21) Appl. No.: 17/253,788

(22) PCT Filed: Jun. 21, 2019
(86) PCT No.: PCT/EP2019/066455

§ 371 Date: Dec. 18, 2020

(30) Foreign Application Priority Data
Jun. 21, 2018 (GB) 1810190.7

(51) Int. Cl. C12Q 1/6886; G16B 25/10; G16B 40/00; C07K 16/28; C07K 16/22; A61K 39/395

(52) U.S. Cl. CPC C12Q 1/6886; G16B 25/10; G16B 40/00; C07K 16/2818; C07K 16/22; A61K 2039/507; A61K 39/3955; C12Q 2600/158; C12Q 2600/106; C07K 2317/76; A61K 39/39541

(57) ABSTRACT

The present invention provides a method for predicting the treatment response to anti-cancer immunotherapy of a mammalian cancer patient, the method comprising: a) measuring the gene expression of at least 2 the following cancer promoting genes: PTGS2, VEGFA, CCL2, IL8, CXCL2, CXCL1, CSF3, IL6, IL1B and IL A in a sample obtained from the tumour of the patient; b) measuring the gene expression of at least 2 of the following cancer inhibitory genes: CXCL11, CXCL10, CXCL9, CCL5, TBX21, EOMES, CD8B, CD8A, PRF1, GZMB, GZMA, STAT1, IFNG, IL12B and IL12A in a sample obtained from the tumour of the patient; c) computing a ratio of the gene expression of said at least 2 cancer promoting genes and the gene expression of said at least 2 cancer inhibitory genes; and d) making a prediction of the treatment response and/or prognosis of the patient based on the gene expression ratio computed in step c). Also provided are related methods for stratifying patients and for treating patients, including with immune checkpoint blockade therapy.

This application claims priority from GB1810190.7, filed 21 Jun. 2018, the contents and elements of which are herein incorporated by reference for all purposes.

### FIELD OF THE INVENTION

The present invention relates to materials and methods for predicting response to cancer therapy and overall survival among cancer patients, particularly patients undergoing immune checkpoint blockade therapy.

### BACKGROUND TO THE INVENTION

The concept that cancer induces inflammation and that inflammatory cells at the tumour site can support cancer progression, is well established (Coussens et al., 2013; Hanahan and Weinberg, 2011; Mantovani et al., 2008). Several cellular and molecular inflammatory mediators commonly found in clinically apparent tumours are well known for having pro-tumourigenic effects and to be associated with many features of aggressive and invasive tumours in both preclinical models and cancer patients. These include prevalent tumour-infiltrating leukocytes such as macrophages, neutrophils, immature myeloid cells or regulatory T cells and molecules produced by these and other leukocytes, stromal cells, or directly by cancer cells. IL-6, IL-8, CCL2, CXCL1 or VEGF are classic examples of soluble factors with pleiotropic effects that can foster cancer growth and spread (Coussens et al., 2013; Mantovani et al., 2008).

Inflammation at the tumour site can also have anti-cancer roles, partly by contributing to immune recognition and elimination of cancer cells. Cytotoxic T cells (CTLs), in particular, are recognised anti-tumour effectors in preclinical cancer models and their intratumoural abundance associates with improved patient outcome and response to cancer therapy (Binnewies et al., 2018; Fridman et al., 2012; Galon, 2006; Thorsson et al., 2018). Accordingly, more favorable prognosis has been also linked with high intratumoural levels of CTL chemoattractants, like CXCL9 or CXCL10, or cytokines that promote type I immunity, CTL differentiation and effector function, such as IL-12 or type I and II interferons (IFNs) (Gajewski et al., 2013; Spranger and Gajewski, 2018; Vesely et al., 2011). The levels of these factors within the tumour microenvironment (TME) can vary depending on the overall systemic and/or local inflammatory status and their integrated effect contributes to the strength and extent of the anti-tumour immune response. In addition to conventional helper CD4+ and cytotoxic CD8⁺ T cells, further evidence indicates that tumour infiltration by other inflammatory cells equally favors immune attack and correlates with good prognosis. Natural killer (NK) cells, gamma delta T cells, innate like lymphocytes and the Batf3-dependent conventional dendritic cells type I (cDC1) constitute some of the immune subsets often associated with improved outcome (Böttcher et al., 2018; Broz et al., 2014; Gentles et al., 2015; Mittal et al., 2017; Morvan and Lanier, 2016; Ruffell et al., 2014; SAnchez-Paulete et al., 2016; Spranger et al., 2015; 2017). This is true both in spontaneous and therapy-induced anti-tumour responses like those ensuing from administration of immune-checkpoint inhibitors, a treatment modality that has revolutionised cancer treatment eliciting beneficial responses, including long-term remissions, in a plethora of cancer types (Ribas and Wolchok, 2018). Especially in immune checkpoint blockade (ICB) therapy, the abundance of select immune cells or inflammatory mediators has been associated positively or negatively with treatment response (Ayers et al., 2017; De Henau et al., 2016; Herbst et al., 2014; Roh et al., 2017; Rooney et al., 2015; Tumeh et al., 2014).

Yet, how antagonistic cancer promoting or inhibitory inflammatory TME are established during cancer development and progression is not understood. Moreover, the signals and pathways that regulate the quality and quantity of the different elements of the inflammatory infiltrate are poorly defined. Improving our understanding of these processes is of great clinical relevance as it could strengthen our ability to predict response from therapy and further provide attractive therapeutic targets to improve the efficacy of anti-cancer treatment.

The cyclooxygenase (COX)-2/prostaglandin E₂ (PGE₂) pathway, upregulated in numerous cancers and implicated in various aspects of malignant growth (Wang and Dubois, 2010), constitutes a candidate fulcrum of the inflammatory phenotype of tumours. In melanoma, colorectal or breast cancer mouse models this pathway plays a dominant role in fueling cancer-promoting inflammation and enabling immune evasion (Zelenay et al., 2015). Accordingly, its genetic ablation in cancer cells impaired their ability to form progressive tumours in immunocompetent, but not immunodeficient, hosts. Tumour growth control, exhibiting unvarying complete remissions in some models, was dependent on cDC1 and adaptive immunity and coupled with a COX-2-driven shift in the intratumoural immune profile characterised by profound alterations in the levels of known cancer-promoting and -inhibitory inflammatory factors (Zelenay et al., 2015).

McDermott et al., Nature Medicine, 2018, Vol. 24, pp. 749-757, describes clinical activity and molecular correlates of response to atezolizumab alone or in combination with bevacizumab versus sunitinib in renal cell carcinoma. Exploratory biomarker analyses indicated that tumour mutation and neoantigen burden were not associated with progression-free survival (PFS). Angiogenesis, T-effector/IFN-γ response, and myeloid inflammatory gene expression signatures were strongly and differentially associated with PFS within and across the treatments.

Mariathasan et al., Nature, 2018, Vol. 554, pp. 544-548, describes TGFβ-mediated attenuation of tumour response to PD-L1 blockade in urothelial cancer. Response to treatment was associated with CD8+ T-effector cell phenotype and, to an even greater extent, high neoantigen or tumour mutation burden. Lack of response was associated with a signature of transforming growth factor β (TGFβ) signalling in fibroblasts. This occurred particularly in patients with tumours, which showed exclusion of CD8+ T cells from the tumour parenchyma that were instead found in the fibroblast- and collagen-rich peritumoural stroma; a common phenotype among patients with metastatic urothelial cancer.

While previously described predictive models of cancer show promise, there remains an unmet need for further models able to predict treatment response and/or survival of cancer patients. The present invention seeks to fulfil these needs and provides further related advantages.

### BRIEF DESCRIPTION OF THE INVENTION

The present inventors used versatile mouse cancer models to define the temporal sequence of events and key immune cell subsets that set the stage for the ensuing T cell-dependent tumour growth control. NK cells were identified as major players for the establishment of a cancer suppressive microenvironment that precedes cDC1- and CTL-mediated tumour eradication. Based on immune gene profiling of these murine tumours with unequivocal progressive or regressive fates, the present inventors derived a COX-2-modulated inflammatory gene signature that shows remarkable power as a biomarker of overall patient survival and of response to anti-PD-1/PD-L1 therapy. Indeed, the COX-2 ratio described herein was found to outperform CD8⁺ T cell, (Spranger et al., 2015), IFN-γ-related (Ayers et al., 2017) and cDC1 gene signatures (Böttcher et al., 2018), underscoring the value of the ‘COX-2 signature’ and the benefit of integrating pro- and anti-tumourigenic factors in a single biomarker.

Accordingly, in a first aspect the present invention provides a method for predicting the treatment response to anti-cancer immunotherapy of a mammalian cancer patient, the method comprising:
a) measuring the gene expression of at least 2, 3, 4, 5, 6, 7, 8, 9 or more (such as all of) the following cancer promoting genes: PTGS2, VEGFA, CCL2, IL8, CXCL2, CXCL1, CSF3, IL6, IL1B and IL1A in a sample obtained from the tumour of the patient;
b) measuring the gene expression of at least 2, 3, 4, 5, 6, 7, 8, 9, 10, 11, 12, 13, 14 or more (such as all of) the following cancer inhibitory genes: CXCL11, CXCL10, CXCL9, CCL5, TBX21, EOMES, CD8B, CD8A, PRF1, GZMB, GZMA, STAT1, IFNG, IL12B and IL12A in a sample obtained from the tumour of the patient;
c) computing a ratio (“COX-2 ratio”) of the gene expression of said at least 2, 3, 4, 5, 6, 7, 8, 9 or 10 cancer promoting genes and the gene expression of said at least 2, 3, 4, 5, 6, 7, 8, 9, 10, 11, 12, 13, 14 or 15 cancer inhibitory genes; and
d) making a prediction of the treatment response and/or prognosis of the patient based on the gene expression ratio computed in step c).

In some embodiments said ratio is of the gene expression of all said cancer promoting genes PTGS2, VEGFA, CCL2, IL8, CXCL2, CXCL1, CSF3, IL6, IL1B and IL1A and all of said cancer inhibitory genes CXCL11, CXCL10, CXCL9, CCL5, TBX21, EOMES, CD8B, CD8A, PRF1, GZMB, GZMA, STAT1, IFNG, IL12B and IL12A.

As described in detail herein, said cancer promoting genes were found to have tumour gene expression that positively correlates with PTGS2 expression, tumour growth and poor treatment response to immunotherapy. Conversely said cancer inhibitory genes were found to have tumour gene expression that negatively correlates with PTGS2 expression, tumour growth and poor treatment response to immunotherapy. The present inventors found that integrating these opposing signals by forming a ratio enhanced predictive power of the gene signature relative to prediction based solely on cancer promoting genes or based solely on cancer inhibitory genes. As the skilled person will be aware, forming a ratio with the gene expression of cancer promoting genes as the numerator and gene expression of cancer inhibitory genes as the denominator means that a higher ratio indicates a worse response to immunotherapy and worse survival time. However, the ratio may alternatively be formed with the gene expression of cancer inhibitory genes as the numerator and gene expression of cancer promoting genes as the denominator. In such an alternative case a lower ratio indicates a worse response to immunotherapy and worse survival time.

In some embodiments said ratio is calculated according to the formula:

$$COX - 2 ratio = \frac{\frac{1}{{n}_{p}} {\sum }_{i = 1}^{{n}_{p}} {G}_{i}^{p o s} ( e )}{\frac{1}{{n}_{n}} {\sum }_{i = 1}^{{n}_{n}} {G}_{i}^{neg} ( e )}$$

wherein nₚ is the number of said cancer promoting genes and nₙ is the number of said cancer inhibitory genes, Gᵢ^{pos} and Gᵢ^{neg} are the positive and negative correlated genes, respectively, within an (i) interval of unitary values, (e) represents the gene expression values, expressed as log 2 counts per million (CPM).

In some embodiments Gᵢ^{pos}(e)=mean expression of log 2 transformed counts per million (Reads Per Kilobase Million (FPKM)) of positive genes and Gᵢ^{neg}(e)=mean expression of log 2 transformed counts per million (FPKM) of negative genes.

In some embodiments COX2 ratio is calculated by dividing Gᵢ^{pos}(e) mean expression of log 2 transformed counts per million (or FPKM) by Gᵢ^{neg}(e) mean expression to give a ratio of cancer promoting and cancer inhibitory genes.

Expression values may be expressed in, for example, any of RPKM (Reads Per Kilobase Million), FPKM (Fragments Per Kilobase Million), CPM (Counts Per Million) and/or nanostring counts.

In some embodiments the expression level of each of said genes is a normalised gene expression level, e.g., normalised to the gene expression of one or more housekeeping gene. In some embodiments the gene expression level may be log-transformed (e.g. log 2-transformed).

In some embodiments the gene expression ratio computed in step c) may be referenced to or compared with the median gene expression ratio of a sample cohort of cancer patients having the same type of cancer as said cancer patient (and optionally age-matched, matched for time since diagnosis and/or matched for disease stage), which median gene expression ratio serves as a threshold, and wherein:
a computed gene expression ratio above said threshold (e.g. 1.1-fold, 1.2-fold or 1.5-fold or more) indicates that said cancer patient is at high risk of a poor treatment response to said anti-cancer immunotherapy and/or at high risk of having a shorter survival time than the median survival time of said sample cohort of cancer patients; and
a computed gene expression ratio below said threshold (e.g. 0.9-fold, 0.8-fold or 0.7-fold or lower) indicates that said cancer patient is at low risk of a poor treatment response to said anti-cancer immunotherapy and/or at low risk of having a shorter survival time than the median survival time of said sample cohort of cancer patients.

In some embodiments said ratio is calculated by:
computing the mean gene expression Z-score for said at least 2 cancer promoting genes and the mean gene expression Z-score for said at least 2 cancer inhibitory genes, wherein said z-score is calculated according to the formula

$$z = \frac{x - μ}{σ}$$

wherein z is the gene expression z-score of a given gene, x is the gene expression of the given gene, μ is the mean expression of the given gene in a training set comprising a plurality of cancer subjects and σ is the standard deviation of the gene expression of the given gene in the training set; and
subtracting the Z-score for said at least 2 cancer inhibitory genes from the Z-score for said at least 2 cancer promoting genes.

In some embodiments said ratio is calculated by:
computing the median gene expression value for each of said at least two cancer promoting genes and said at least two cancer inhibitory genes across a training set comprising a plurality of cancer subjects,
applying, for each of said genes, a value of +1 where the expression value of said cancer patient is greater than the median of that gene over the training set,
summing the cancer inhibitory gene scores and summing the cancer promoting gene scores, and
subtracting the summed cancer inhibitory gene score from the summed cancer promoting gene score, optionally after normalising to account for the number of cancer inhibitory genes and the number of cancer promoting genes, respectively. For example, if the cancer promoting (CP) signature has 10 genes and the cancer inhibitory (CI) signature has 15 genes, the CP signature score may be multiplied by 15/10 in order to normalise it up to the higher number of CI genes.

In some embodiments said ratio is calculated by:
computing the mean gene expression Z-score for said at least 2 cancer promoting genes and the mean gene expression Z-score for said at least 2 cancer inhibitory genes wherein said z-score is calculated according to the formula

$$z = \frac{x - μ}{σ}$$

wherein z is the gene expression z-score of a given gene, x is the gene expression of the given gene, μ is the mean expression of the given gene in a training set comprising a plurality of cancer subjects and σ is the standard deviation of the gene expression of the given gene in the training set;
applying, for each of said genes, a value of +1 where the z-score is greater than 0.1, a value of −1 where the z-score is less than −0.1, and a value of 0 where the z-score is between 0.1 and −0.1;
summing the cancer inhibitory gene applied values and summing the cancer promoting gene applied values, and
subtracting the summed cancer inhibitory gene applied values from the summed cancer promoting gene applied values.

In some embodiments said ratio is calculated by:
computing the mean gene expression Z-score for said at least 2 cancer promoting genes and the mean gene expression Z-score for said at least 2 cancer inhibitory genes wherein said z-score is calculated according to the formula

$$z = \frac{x - μ}{σ}$$

wherein z is the gene expression z-score of a given gene, x is the gene expression of the given gene, μ is the mean expression of the given gene in a training set comprising a plurality of cancer subjects and σ is the standard deviation of the gene expression of the given gene in the training set;
applying, for each of said genes, a value of +1 where the z-score is greater than 0.3, a value of −1 where the z-score is less than −0.3, and a value of 0 where the z-score is between 0.3 and −0.3;
summing the cancer inhibitory gene applied values and summing the cancer promoting gene applied values, and
subtracting the summed cancer inhibitory gene applied values from the summed cancer promoting gene applied values. In some embodiments, the method comprises normalising to account for the number of cancer inhibitory genes and the number of cancer promoting genes, respectively. For example, if the cancer promoting (CP) signature has 10 genes and the cancer inhibitory (CI) signature has 15 genes, the CP signature score may be multiplied by 15/10 in order to normalise it up to the higher number of CI genes.

In some embodiments the method further comprises assessing other tumour features likely to add benefit to the predictive power of the COX-2 ratio, such as the tumour burden and/or neoantigen prevalence of the cancer patient.

In some embodiments the cancer may be a solid tumour. In particular, the cancer may be melanoma (e.g. metastatic melanoma), renal cancer (e.g. sarcomatoid or clear cell renal cell carcinoma), or bladder cancer (e.g. metastatic urothelial carcinoma). As described herein, the COX-2 ratio was found to strongly associate with treatment response outcomes in datasets relating to melanoma, to bladder cancer and renal cell carcinoma. The COX-2 ratio was predictive regardless of how the two gene signatures were calculated and combined together, the method was equally able to distinguish patients with divergent clinical responses and overall survival. The method was found to hold independent predictive and prognostic power in multiple cohorts when it was combined with typical clinical parameters such as staging, as well as when combined with published genomic and transcriptomic biomarkers such as tumour mutational burden, PD-L1 immunohistochemistry and other gene signatures.

In certain embodiments, where the gene expression ratio computed in step c) indicates that the cancer patient is predicted to respond to anti-cancer immunotherapy, the method may further comprise selecting the cancer patient for anti-cancer immunotherapy. In particular, said anti-cancer immunotherapy may comprise immune checkpoint blockade therapy. Exemplary immune checkpoint blockade therapy comprises programmed death-1 (PD-1) blockade, programmed death-ligand 1 (PD-L1) blockade and/or cytotoxic T-lymphocyte-associated protein 4 (CTLA-4) blockade. Examples of agents (e.g. monoclonal antibodies) considered immune checkpoint blockade therapies include Nivolumab, Pembrolizumab, Atezolizumab and/or Ipilimumab.

In a second aspect, the present invention provides a method of stratifying a plurality of cancer patients according to their method predicted response to anti-cancer immunotherapy, the method comprising carrying out the method of the first aspect of the invention on each of said plurality of cancer patients.

In a third aspect, the present invention provides a computer-implemented method for predicting the treatment response to anti-cancer immunotherapy of a mammalian cancer patient, the method comprising:
a) providing gene expression data comprising expression levels of at least 2, 3, 4, 5, 6, 7, 8, 9 or more (such as all of) the following cancer promoting genes: PTGS2, VEGFA, CCL2, IL8, CXCL2, CXCL1, CSF3, IL6, IL1B and IL1A previously measured in a sample obtained from the tumour of the patient;
b) providing gene expression data comprising expression levels at least 2, 3, 4, 5, 6, 7, 8, 9, 10, 11, 12, 13, 14 or more (such as all of) the following cancer inhibitory genes: CXCL11, CXCL10, CXCL9, CCL5, TBX21, EOMES, CD8B, CD8A, PRF1, GZMB, GZMA, STAT1, IFNG, IL12B and IL12A in a sample obtained from the tumour of the patient;
c) computing a ratio of the gene expression of said at least 2, 3, 4, 5, 6, 7, 8, 9 or 10 cancer promoting genes and the gene expression of said at least 2, 3, 4, 5, 6, 7, 8, 9, 10, 11, 12, 13, 14 or 15 cancer inhibitory genes;
d) comparing the computed ratio from step c) with a reference median gene expression ratio derived from a sample cohort of cancer patients having the same type of cancer as said cancer patient; and
e) making a prediction of the treatment response and/or prognosis of the cancer patient based on the comparison made in step d).

In some embodiments the gene expression data may have been pre-determined and/or may be provided by retrieval from a volatile or non-volatile computer memory or data store (including cloud storage).

In some embodiments said ratio is of the gene expression of all said cancer promoting genes PTGS2, VEGFA, CCL2, IL8, CXCL2, CXCL1, CSF3, IL6, IL1B and IL1A and all of said cancer inhibitory genes CXCL11, CXCL10, CXCL9, CCL5, TBX21, EOMES, CD8B, CD8A, PRF1, GZMB, GZMA, STAT1, IFNG, IL12B and IL12A.

In some embodiments said ratio is calculated according to the formula:

$$COX - 2 ratio = \frac{\frac{1}{{n}_{p}} {\sum }_{i = 1}^{{n}_{p}} {G}_{i}^{pos} ( e )}{\frac{1}{{n}_{n}} {\sum }_{i = 1}^{{n}_{n}} {G}_{i}^{neg} ( e )}$$

wherein nₚ is the number of said cancer promoting genes and nₙ is the number of said cancer inhibitory genes, Gᵢ^{pos} and Gᵢ^{neg} are the positive and negative correlated genes, respectively, within an (i) interval of unitary values, (e) represents the gene expression values, expressed as log 2 counts per million (CPM).

In some embodiments Gᵢ^{pos}(e)=mean expression of log 2 transformed counts per million (Reads Per Kilobase Million (FPKM)) of positive genes and Gᵢ^{neg}(e)=mean expression of log 2 transformed counts per million (FPKM) of negative genes.

In some embodiments COX2 ratio is calculated by dividing Gᵢ^{pos}(e) mean expression of log 2 transformed counts per million (or FPKM) by Gᵢ^{neg}(e) mean expression to give a ratio of cancer promoting and cancer inhibitory genes.

Expression values may be expressed in, for example, any of RPKM (Reads Per Kilobase Million), FPKM (Fragments Per Kilobase Million), CPM (Counts Per Million) and/or nanostring counts.

In some embodiments the expression level of each of said genes is a normalised gene expression level and/or a log-transformed (e.g. log 2-transformed) gene expression level.

In some embodiments, said ratio is calculated as defined for any embodiment of the first aspect of the invention.

In a fourth aspect, the present invention provides a method of treatment of a cancer in a mammalian patient, comprising:
(a) carrying out the method of the first aspect of the invention;
(b) determining that the gene expression ratio computed in step c) indicates that the cancer patient is predicted to respond to anti-cancer immunotherapy; and
(c) administering immunotherapy (e.g. immune checkpoint blockade therapy) to the patient in need thereof.

In some embodiments said immune checkpoint blockade therapy comprises programmed death-1 (PD-1) blockade, programmed death-ligand 1 (PD-L1) blockade and/or cytotoxic T-lymphocyte-associated protein 4 (CTLA-4) blockade.

In some embodiments said immune checkpoint blockade therapy comprises treatment with a therapeutically effective amount of Nivolumab, Pembrolizumab, Atezolizumab and/or Ipilimumab.

In some embodiments in accordance with any aspect of the present invention, immune checkpoint blockade therapy may be combined with anti-angiogenesis therapy, such as anti-vascular endothelial growth factor (anti-VEGF) therapy (e.g. Bevacizumab). As shown in detail herein (see, e.g., FIGS. 11G and 11H) COX-IS was found to be significantly different between non-responders (NR) and responders (R) in the group treated with a combination of anti-PD-L1 antibody and anti-VEGFA antibody.

In accordance with any aspect of the present invention, the subject may be a human, a companion animal (e.g. a dog or cat), a laboratory animal (e.g. a mouse, rat, rabbit, pig or non-human primate), a domestic or farm animal (e.g. a pig, cow, horse or sheep).

Preferably, the subject is a human patient. In some cases the patient may be a plurality of patients. In particular, the methods of the present invention may be for stratifying a group of patients (e.g. for a clinical trial) into high and low risk or into high, moderate and low risk subgroups based on their gene expression profiles.

Embodiments of the present invention will now be described by way of example and not limitation with reference to the accompanying figures. However various further aspects and embodiments of the present invention will be apparent to those skilled in the art in view of the present disclosure.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1: Ablation of cancer cell-intrinsic COX alters the intratumoural accumulation of select innate immune cell subsets. (A) Tumour growth profile of Ptgs^{+/+}, Ptgs^{-/-} and Ptgs^{-/-}+COX-2 Braf^{V600E} melanoma cells (1×10⁵) injected sc in immune competent mice. (B) PGE₂ levels in supernatant and COX-2 protein expression in Ptgs^{+/+}, Ptgs^{-/-} and Ptgs^{-/-}+COX-2 Braf^{V600E} melanoma cells. (C) Tumour weight of Ptgs^{+/+}, Ptgs^{-/-} and Ptgs^{-/-}+COX Braf^{V600E} melanomas analysed 4 days after cell injection (2×10⁶, sc). (D and E) Tumour infiltrate analysed by flow cytometry 4 days after Ptgs^{+/+}, Ptgs^{-/-} and Ptgs^{-/-}+COX-2 Braf^{V600E} melanoma injection (2×10⁶ cells, sc). The frequency and the number of intratumoural neutrophils (CD11b⁺ Ly6G⁺) (D) and NK cells (NK1.1+) (E) are shown. Immunofluorescence analysis of neutrophils (F) and NK cells (G) in Ptgs^{+/+} and Ptgs^{-/-} melanomas harvested 4 days after cell injection. Data are expressed as mean±SEM. *p<0.05, **p<0.01, ***p<0.001, paired (A) or unpaired (C to G) Student's t test.

FIG. S1: Genetic ablation of COX alters the intratumoural accumulation of innate immune cells. (A) Representative gating strategy. (B to F) Tumour infiltrate analysed by flow cytometry 4 days after Ptgs^{+/+}, Ptgs^{-/-} and Ptgs^{-/-}+COX Braf^{V600E} melanoma injection (2×10⁶, sc). The frequency and the number of intratumoural monocytes (CD11b⁺ Ly6C⁺) (B), macrophages (CD11b⁺ F4/80⁺ Ly6G⁻ Ly6C⁻) (C), eosinophils (D) CD11c⁺ MHC II⁺ cells (E) and cDC₁ (E) are shown. (G) Kinetic of neutrophil (left panel) and NK cell (right panel) accumulation in Ptgs^{+/+} and Ptgs^{-/-} Braf^{V600E} melanoma tumours at indicated time points, after tumour cell inoculation (2×10⁶, sc). (H) Frequencies of tumour infiltrating leukocytes on day 30 post tumour cell inoculation (1×10⁵, sc). Frequencies are shown as percentage of parental gate. Neutrophils, NK cells, CD3⁺ cells and CD11c⁺ MHCII⁺ cells are gated out of CD45⁺ cells; CD8⁺ T cells are gated on CD3⁺ cells and cDC1 are gated on CD11c⁺ MHCII⁺ cells. Data are expressed as mean±SEM. *p<0.05, **p<0.01, ***p<0.001, unpaired Student's t test.

FIG. 2: Neutrophil and NK cell accumulation within the TME is regulated by cancer cell-intrinsic COX activity independently of cancer type. (A) PGE₂ levels in supernatant and COX-2 protein expression in Ptgs^{+/+}, Ptgs^{-/-} and Ptgs^{-/-}+COX-2 MC38 colorectal cancer cells. (B) Tumour growth profile of Ptgs^{+/+}, Ptgs^{-/-} and Ptgs^{-/-}+COX MC38 colorectal cancer cells (1×10⁵) injected s.c. in immune competent, Rag1^{-/-} and Batf3^{-/-} mice. (C) Tumour weight of Ptgs^{+/+}, Ptgs^{-/-} MC38 colorectal cancer (red), 4T1 breast cancer (green) and CT26 colorectal cancer (blue) analysed 4 days after cell injection (2×10⁶, sc). (D) Tumour infiltrate analysed by flow cytometry 4 days after Ptgs^{+/+}, Ptgs^{-/-} and Ptgs^{-/-}+COX MC38 colorectal cancer (red), Ptgs^{+/+} and Ptgs^{-/-} 4T1 breast cancer (green) and Ptgs^{+/+} and Ptgs^{-/-} CT26 colorectal cancer (blue) cell injection (2×10⁶, s.c.). The frequencies of intratumoural neutrophil (upper panels) and NK cell populations (lower panels) are shown. Data are expressed as mean±SEM. *p<0.05, **p<0.01, ***p<0.001, paired (B) or unpaired (C and D) Student's t test.

FIG. S2: Accumulation of neutrophils and NK cells within the TME is controlled by COX activity independently of tumour type. Tumour infiltrate analysed by flow cytometry 4 days after Ptgs^{+/+}, Ptgs^{-/-} and Ptgs^{-/-}+COX-2 MC38 colorectal cancer (red), Ptgs^{+/+} and Ptgs^{-/-} 4T1 breast cancer (green) and Ptgs^{+/+} and Ptgs^{-/-} CT26 colorectal cancer (blue) cell injection (2×10⁶, sc). The frequency of total infiltrating leukocytes (A) and all other innate immune cell populations analysed (B) is shown. Data are expressed as mean±SEM. *p<0.05, **p<0.01, ***p<0.001, unpaired Student's t test.

FIG. 3: NK cell-depletion abolishes spontaneous and ICB-induced control of tumour growth. NK cell and neutrophil frequencies in Ptgs^{/++} and Ptgs^{-/-} Braf^{V600E} melanoma (A) and MC38 colorectal cancer (D) tumours (2×10⁶ cells, sc) in immune competent mice after NK cell depletion. Tumour weight of Ptgs^{+/+} and Ptgs^{-/-} Braf^{V600E} melanoma (B) and MC38 colorectal cancer (E) analysed 4 days after tumour cell injection (2×10⁶ cells, sc). Tumour growth profile of Ptgs^{-/-} Braf^{V600E} melanoma (C) and MC38 colorectal cancer (F) cells (1×10⁵ cells, sc) in immune competent mice receiving NK cell-depleting antibodies. (G) Tumour growth profiles of Ptgs^{+/+} and Ptgs^{-/-} Braf^{V600E} melanoma cells (1×10⁵ cells, sc) inoculated in immune competent mice receiving NK cell-, CD4⁺ cell-, CD8⁺ cell-depleting antibodies, in Rag1^{-/-} or in Batf3^{-/-} mice. (H) Comparison of individual grow profiles of Ptgs^{-/-} Braf^{V600E} melanoma cells in immune competent mice receiving NK cell-depleting antibodies from one day before (turquoise) or seven days after (blue) cell inoculation. (I) Tumour growth profiles and (J) tumour diameter (day 20) of Ptgs^{+/+} Braf^{V600E} melanoma cells (1×10⁵ injected sc) in immune competent mice treated with vehicle (black), anti-PD-1 (200 μg i.p./twice weekly)+celecoxib (30 mg/kg daily) (red) or anti-PD-1+celecoxib in mice depleted of NK cells (blue). (K) Survival analysis of anti-PD-1+celecoxib treated mice vs anti-PD-1+celecoxib treated/NK cells depleted mice. Data are expressed as mean±SEM. *p<0.05, **p<0.01, ***p<0.001, paired (C, F and G) or unpaired (A, B, D, E and J) Student's t test. **p<0.01, ***p<0.001, Log-rank test (K).

FIG. S3: NK cell depletion does not alter the accumulation of other innate immune populations in tumours. (A) Tumour weight, total leukocyte, neutrophil, and NK cell frequencies in melanoma tumours analysed 4 days after cell transplantation in mice receiving anti-GR-1 antibodies. (B) Monocyte (CD11b⁺ Ly6C⁺), TAM (CD11b⁺ F4/80⁺ Ly6G⁻ Ly6C⁻), CD11c⁺ MHC II⁺ cells and cDC₁ frequencies in melanoma tumours analysed 4 days after cell transplantation in mice receiving anti-GR-1 antibodies. (C) Monocyte, TAM, CD11c⁺ MHC II⁺ cells and cDC₁ frequencies Ptgs^{+/+} and Ptgs^{-/-} Braf^{V600E} melanoma (black) and MC38 colorectal cancer (red) analysed 4 days after cell transplantation in mice receiving NK-cell depleting antibodies. Data are expressed as mean±SEM. *p<0.05, **p<0.01, ***p<0.001, unpaired Student's t test.

FIG. 4: NK cells drive reprogramming of the TME toward type I immunity. (A) Analysis by RT-PCR of bulk Ptgs^{+/+} and Ptgs^{-/-} Braf^{V600E} melanoma tumours after NK cell depletion. Tumours were analysed 4 days after cell inoculation. Markers associated with cancer promoting (red) and inhibitory inflammation (blue) are shown. Data were relative to hprt expression and displayed in the heatmap as row Z-Score. (B and C) Analysis by RT-PCR of cancer inhibitory genes (B), cd3e and cDC1-related molecules (C) in Ptgs^{+/+} (N=12) and Ptgs^{-/-} (N=12) Braf^{V600E} melanoma tumours from mice depleted of NK cells (N=9), Rag1^{-/-} (N=9) and Batf3^{-/-} (N=9). Data were relative to hprt, normalised on the average expression of Ptgs^{+/+} tumours and expressed as mean±SEM. *p<0.05, **p<0.01, ***p<0.001, unpaired Student's t test. All the comparison are vs Ptgs^{-/-} tumours. (D) FACS analysis of DC activation marker CD40 and CD86 expression on cDC1 and cDC1 frequency from Ptgs^{+/+} and Ptgs^{-/-} Braf^{V600E} melanoma tumours after NK cell depletion. Data are expressed as mean±SEM. ***p<0.001, unpaired Student's t test.

FIG. 5: COX-2 expression delineates cancer-promoting from cancer-inhibitory inflammation in human cancers. (A) Correlation analysis of PTGS2 versus a NK-cell driven, mouse-derived inflammatory gene signature in TCGA datasets: LUAD (n=522), HNSC (n=530), TN_BRCA (n=320), UCEC (n=548), MESO (n=87), P_SKCM (n=119), KIRC (n=538), M_SKCM (n=360), LUSC (n=504), STAD (n=295), ESCA (n=186), PDAC (n=186), BLCA (n=413), HER2_BRCA (n=184), LAML (n=200), ER_BRCA (n=1165), PRAD (n=499), COAD (n=633), LIHC (n=442) and UVMM (n=80). The heatmap shows the positive (red) or negative (blue) Pearson correlation coefficient between PTGS2 and the indicated genes. (B) Correlation plots of COX-2 signature in LUAD and HNSC datasets. The Pearson coefficient and the p value for individual genes is shown. (C) Correlation analysis between PTGS2 and a neutrophil or NK cell signature (see methods) in LUAD and HNSC datasets.

FIG. 6: The COX-2 signature strongly associates with patient prognosis and immune cell tumour infiltrate composition in human cancer. (A) Survival analysis of LAUD (n=522), HNSC (n=530), TN_BRCA (n=320) and M_SKCM (n=360) patients stratified accordingly to COX-2 ratio. Kaplan-Maier plots data are parsed as high (red genes/blue genes) ratio versus low (red genes/blue genes) ratio expressers. Patient overall survival was compared by Log-rank (Mantel-Cox) test. (B) Analysis of the individual contribution of each gene included in COX-2 signature and comparison with previously published signatures. (C and D) Comparison of immune cell fractions among COX-2 ratio-high and -low patients in LAUD (n=522), HNSC (n=530), TN_BRCA (n=320) and M_SKCM (n=360) datasets. Immune cell fractions were estimated using CIBERSORT algorithm corrected for RNAseq data. Data are expressed as mean±SEM. *p<0.05, **p<0.01, ***p<0.001, unpaired Student's t test.

FIG. S6: COX-2 ratio-based patient stratification delineates tumours with different immune cell composition. (A) CD8⁺ T cell-Treg ratio. Values calculated using CIBERSORT algorithm. (B) Comparison of immune cell fractions in COX-2 ratio-high and -low patients in LAUD (n=522), HNSC (n=530), TN_BRCA (n=320) and M_SKCM (n=360) datasets. Immune cell fractions were estimated using xCELL algorithm. (C) Summary heatmaps of immune population infiltrating LUAD, HNSC, TN_BRCA and M_SKCM calculated using xCELL algorithm in patients stratified accordingly to COX-2 ratio. Data are expressed as mean±SEM. *p<0.05, **p<0.01, ***p<0.001, unpaired Student's t test.

FIG. 7: The COX-2 ratio predicts response to PD-1 and PD-L1 blockade. Analysis of COX-2 ratio (A) at baseline in melanoma (Riaz et al., Chen et al., and Roh et al.) and bladder cancer (Mariathasan et al.) patients receiving anti-PD-1 or anti PD-L1 treatments respectively. R=responder, NR=non-responder, NPD=non-progressive disease, PD=progressive disease. (B) Survival analysis of patients from Riaz et al., and Mariathasan et al. stratified on the median value of COX-2 ratio, cancer promoting siganture and cancer inhibitory signature. Kaplan-Maier plots data are parsed as high (red genes/blue genes) ratio versus low (red genes/blue genes) ratio expressers. Patient overall survival was compared by Log-rank (Mantel-Cox) test. (C) COX-2 ratio value in PD=progressive disease, SD=stable disease, PR=partial response, CR=complete response patients from Mariathasan et al. (D) Patient overall survival from Mariathasan et al. stratified on COX-2 ratio in quartiles.

FIG. S7: The COX-2 ratio predicts response to PD-1 and PD-L1 blockade. (A) Analysis of the individual contribution of each gene included in COX-2 signature in melanoma (Riaz et al., Chen et al., and Roh et al.) and bladder cancer (Mariathasan et al.) datasets. (B) Survival analysis of patients from Riaz et al., and Mariathasan et al. stratified on the median value of T cell, IFNγ and cDC1 signatures. Kaplan-Maier plots data are parsed as high (red genes/blue genes) ratio versus low (red genes/blue genes) ratio expressers. Patient overall survival was compared by Log-rank (Mantel-Cox) test.

FIG. 8: The COX-2 signature strongly associates with patient survival independently of tumour-infiltrating CD8⁺ T cell abundance. (A and B) Survival analysis of LUAD (n=512), HNSC (n=517), TNBC (n=320), MSKCM (n=357) and CESC (n=305) patients stratified according to the COX-IS (see methods). (A) Kaplan-Maier survival (KM) plots parsed as high versus low COX-IS expressers at a 75% (LUAD and CESC) or 50% (HNSC, TNBC and MSKCM) stringency. (B) Hazard ratio associated with the indicated gene signatures or the individual gene elements of the COX-IS. (C-D) KM plots of IFNγ dominant patients (n=2884) from all TCGA samples stratified for cancer inhibitory (CI) signature (C) or for the combination of cancer promoting (CP)/CI (D) signatures defined as high or low according the median. (E) CD8⁺ T cells score based on CD8A, CD8B and CD3E expression in the patient subsets shown in D. Hazard ratio (95% C.I.), Log-rank (Mantel-Cox) test (A-D).

FIG. 9: COX-IS is an independent prognostic factor across selected cancer types. (A) Forest plot showing hazard ratios, and associated confidence intervals, from multivariate Cox regression analysis in LUAD, HNSC, MSKCM, TNBC and CESC datasets. Stage was converted to a continuous variable. Sex indicates the relative risk for males against females. COX-IS indicates the relative risk for low versus high COX-IS patients. For HNSC, HPV state compares positive versus negative patients. For CESC each histological subtype is compared to mucinous carcinoma. (B) Survival analysis of all TCGA patients (n=10718) stratified according to the COX-IS. Kaplan-Maier plots data are parsed as high versus low COX-IS expressers (50% stringency).

FIG. 10: The COX-IS predicts response from immune checkpoint blockade across different tumour types. (A) Analysis of COX-IS at baseline in responder (R) and non-responder (NR) groups in melanoma (dataset #1: Riaz et al., #2: Van Allen et al., #3: Hugo et al., #4: Gide et al.), bladder (dataset #5: Mariathasan et al., #6: Snyder et al.), renal (dataset #7: McDermott et al.) and gastric (dataset #8: Kim et al.) cancer patients as defined in the original studies (see methods). (B) Paired analysis of COX-IS in R and NR patients shown in A. (C) Survival of patients from dataset #5 stratified in quantiles according to their COX-IS. (D and H) COX-IS in progressive disease (PD), stable disease (SD), partial response (PR) and complete response (CR) patient groups from dataset #5 and #7. (E, F, I and J) Explained variance (deviance) in patient response for generalised linear models fit using single indexes (E and I) or their combinations (F and J) as input variables. Chi-squared test was used to compare nested models. As single index, COX-IS explained a significant level of variation in patient response both in dataset #5 and #7 (E and I). (G and K) Violin plots of Cohen's Kappa from 10-fold (G) or 5-fold (K) cross validation with 100 repeats. The SMOTE method was used to balance the classes within re-sampling (see methods). Kappa values from cross-validation are shown.

FIG. 11: The COX-IS predicts response from immune checkpoint blockade across different tumour types. (A) Survival of patients from dataset #5 stratified in quantiles according to their NK cell abundance defined as high or low according the median. (B) Multivariate Cox regression analysis for dataset #5. COX-IS indicates the hazard ratio comparing low versus high COX-IS. Sex refers to males compared to females. Both visceral and liver metastasis indicates the relative risk against those with lymph node only metastasis. (C) Receiver operating characteristic (ROC) analysis for the indicated parameters in PD vs CR patients from dataset #5. (D) Kaplan-Mayer_survival plots of melanoma patients from all datasets combined stratified in 4 quantiles according to their COX-IS. (E) Forest plot showing multivariate Cox regression analysis for the combined melanoma datasets. (F) Kaplan-Mayer_survival plots of melanoma patients from dataset #2 stratified for CP, CI or COX-IS. (G) Analysis of COX-IS at baseline in R and NR groups from dataset #7. Patients were divided according to the treatment received in TKI (sunitinib treated), anti-PD-L1 (single agent) or anti-PD-L1 combined with anti-VEGFA (bevacizumab). (H) ROC analysis for the indicated parameters in PD vs CR patient in dataset #7. The area under the ROC curve (AUC) was used to quantify response prediction. Patient overall survival was compared by Log-rank (Mantel-Cox) test.

FIG. 12. Comparison of alternative methods for calculation of the COX-2 ratio. COX-IS score for non-responders (NR) and responders (R) for dataset #5 (Mariathasan et al.) and dataset #7 (McDermott et al.) calculated with Methods 1, 2, 3, 4 and 5.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

In describing the present invention, the following terms will be employed, and are intended to be defined as indicated below.

Samples

A “test sample” as used herein may be a cell or tissue sample (e.g. a biopsy), a biological fluid, an extract (e.g. a protein or DNA extract obtained from the subject). In particular, the sample may be a tumour sample, e.g. a solid tumour such as a gastroesophageal tumour, a melanoma, a bladder tumour or a renal tumour. The sample may be one which has been freshly obtained from the subject or may be one which has been processed and/or stored prior to making a determination (e.g. frozen, fixed or subjected to one or more purification, enrichment or extractions steps).

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

COX-2 Ratio

As used herein the terms “COX-2 ratio”, “COX-IS” and “Inflammatory Score Associated with Cyclooxygenase” (“ISAC”) are used interchangeably. As described in detail herein, cancer promoting genes (see Table 1) were found to have tumour gene expression that positively correlates PTGS2 expression, tumour growth and poor treatment response to immunotherapy. Conversely cancer inhibitory genes (see Table 2) were found to have tumour gene expression that negatively correlates with PTGS2 expression, tumour growth and poor treatment response to immunotherapy. The present inventors found that integrating these opposing signals by forming a ratio enhances the predictive power of the gene signature relative to prediction based solely on cancer promoting genes or based solely on cancer inhibitory genes. As used herein the term “ratio” such as in “COX-2 ratio” is intended to have a broad meaning, not only encompassing one value divided by another, but also to include any relationship that combines the opposing signals, such as one score subtracted from the other (e.g. a difference between CI and CP gene expression scores).

TABLE 1
Cancer promoting genes

| | | NCBI Gene ID* |
|---|---|---|
| | | for human |
| Gene Symbol | Gene Name | gene |
| PTGS2 | prostaglandin-endoperoxide synthase 2 | 5743 |
| VEGFA | vascular endothelial growth factor A | 7422 |
| CCL2 | C-C motif chemokine ligand 2 | 6347 |
| IL8 (CXCL8) | C-X-C motif chemokine ligand 8 | 3576 |
| CXCL2 | C-X-C motif chemokine ligand 2 | 2920 |
| CXCL1 | C-X-C motif chemokine ligand 1 | 2919 |
| CSF3 | colony stimulating factor 3 | 1440 |
| IL6 | interleukin 6 | 3569 |
| IL1B | interleukin 1 beta | 3553 |
| IL1A | interleukin 1 alpha | 3552 |

*NCBI Gene ID (version as of 17 June 2018). Available at https://www.ncbi.nlm.nih.gov/gene. The nucleotide sequence for each gene as disclosed at that NCBI Gene ID number on 17 June 2018 is expressly incorporated herein by reference.

TABLE 2
Cancer inhibitory genes

| | | NCBI Gene ID* |
|---|---|---|
| | | for human |
| Gene Symbol | Gene Name | gene |
| CXCL11 | C-X-C motif chemokine ligand 11 | 6373 |
| CXCL10 | C-X-C motif chemokine ligand 10 | 3627 |
| CXCL9 | C-X-C motif chemokine ligand 9 | 4283 |
| CCL5 | C-C motif chemokine ligand 5 | 6352 |
| TBX21 | T-box 21 | 30009 |
| EOMES | eomesodermin | 8320 |
| CD8B | CD8b molecule | 926 |
| CD8A | CD8a molecule | 925 |
| PRF1 | perforin 1 | 5551 |
| GZMB | granzyme B | 3002 |
| GZMA | granzyme A | 3001 |
| STAT1 | signal transducer and activator of | 6772 |
| | transcription 1 | |
| IFNG | interferon gamma | 3458 |
| IL12B | interleukin 12B | 3593 |
| IL12A | interleukin 12A | 3592 |

*NCBI Gene ID (version as of 17 June 2018). Available at https://www.ncbi.nlm.nih.gov/gene. The nucleotide sequence for each gene as disclosed at that NCBI Gene ID number on 17 June 2018 is expressly incorporated herein by reference.

In some embodiments the COX-2 ratio (also known as COX-IS/ISAC) is calculated according to the formula:

$$COX - 2 ratio = \frac{\frac{1}{{n}_{p}} {\sum }_{i = 1}^{{n}_{p}} {G}_{i}^{pos} ( e )}{\frac{1}{{n}_{n}} {\sum }_{i = 1}^{{n}_{n}} {G}_{i}^{neg} ( e )}$$

wherein nₚ is the number of said cancer promoting genes and nₙ is the number of said cancer inhibitory genes, Gᵢ^{pos} and Gᵢ^{neg} are the positive and negative correlated genes, respectively, within an (i) interval of unitary values, (e) represents the gene expression values, expressed as log 2 counts per million (CPM).

In some embodiments Gᵢ^{pos}(e)=mean expression of log 2 transformed counts per million (Reads Per Kilobase Million (FPKM)) of positive genes and Gᵢ^{neg}(e)=mean expression of log 2 transformed counts per million (FPKM) of negative genes.

In some embodiments COX2 ratio is calculated by dividing Gᵢ^{pos}(e) mean expression of log 2 transformed counts per million (or FPKM) by Gᵢ^{neg}(e) mean expression to give a ratio of cancer promoting and cancer inhibitory genes.

Expression values may be expressed in, for example, any of RPKM (Reads Per Kilobase Million), FPKM (Fragments Per Kilobase Million), CPM (Counts Per Million) and/or nanostring counts.

Gene Expression

Reference to determining the expression level refers to determination of the expression level of an expression product of the gene. Expression level may be determined at the nucleic acid level or the protein level.

The gene expression levels determined may be considered to provide an expression profile. By “expression profile” is meant a set of data relating to the level of expression of one or more of the relevant genes in an individual, in a form which allows comparison with comparable expression profiles (e.g. from individuals for whom the prognosis is already known), in order to assist in the determination of prognosis and in the selection of suitable treatment for the individual patient.

The determination of gene expression levels may involve determining the presence or amount of mRNA in a sample of cancer cells. Methods for doing this are well known to the skilled person. Gene expression levels may be determined in a sample of cancer cells using any conventional method, for example using nucleic acid microarrays or using nucleic acid synthesis (such as quantitative PCR). For example, gene expression levels may be determined using a NanoString nCounter Analysis system (see, e.g., U.S. Pat. No. 7,473,767).

Alternatively or additionally, the determination of gene expression levels may involve determining the protein levels expressed from the genes in a sample containing cancer cells obtained from an individual. Protein expression levels may be determined by any available means, including using immunological assays. For example, expression levels may be determined by immunohistochemistry (IHC), Western blotting, ELISA, immunoelectrophoresis, immunoprecipitation, flow cytometry, mass cytometry and immunostaining. Using any of these methods it is possible to determine the relative expression levels of the proteins expressed from the genes listed in Tables 1 and 2.

Gene expression levels and the ratio derived therefrom as detailed herein may be compared with the expression levels and corresponding ratio of the same genes in cancers from a group of patients whose survival time and/or treatment response is known. The patients to which the comparison is made may be referred to as the ‘control group’. Accordingly, the determined gene expression levels and ratio may be compared to the expression levels in a control group of individuals having cancer. The comparison may be made to expression levels determined in cancer cells of the control group. The comparison may be made to expression levels determined in samples of cancer cells from the control group. The cancer in the control group may be the same type of cancer as in the individual. For example, if the expression is being determined for an individual with melanoma, the expression levels and ratio may be compared to the expression levels and ratio in the cancer cells of patients also having melanoma.

Other factors may also be matched between the control group and the individual and cancer being tested. For example, the stage of cancer may be the same, the subject and control group may be age-matched and/or gender matched.

Additionally, the control group may have been treated with the same form of surgery and/or same chemotherapeutic treatment.

Accordingly, an individual may be stratified or grouped according to their similarity of gene expression ratio with the group with good or poor prognosis, respectively.

Methods for Classification Based on Gene Expression

In some embodiments, the present invention provides methods for classifying, prognosticating, or monitoring cancer in subjects. In particular, data obtained from analysis of gene expression may be evaluated using one or more pattern recognition algorithms. Such analysis methods may be used to form a predictive model, which can be used to classify test data. For example, one convenient and particularly effective method of classification employs multivariate statistical analysis modelling, first to form a model (a “predictive mathematical model”) using data (“modelling data”) from samples of known subgroup (e.g., from subjects known to have a particular cancer prognosis subgroup: high risk and low risk), and second to classify an unknown sample (e.g., “test sample”) according to subgroup.

Pattern recognition methods have been used widely to characterise many different types of problems ranging, for example, over linguistics, fingerprinting, chemistry and psychology. In the context of the methods described herein, pattern recognition is the use of multivariate statistics, both parametric and non-parametric, to analyse data, and hence to classify samples and to predict the value of some dependent variable based on a range of observed measurements. There are two main approaches. One set of methods is termed “unsupervised” and these simply reduce data complexity in a rational way and also produce display plots which can be interpreted by the human eye. However, this type of approach may not be suitable for developing a clinical assay that can be used to classify samples derived from subjects independent of the initial sample population used to train the prediction algorithm.

The other approach is termed “supervised” whereby a training set of samples with known class or outcome is used to produce a mathematical model which is then evaluated with independent validation data sets. Here, a “training set” of gene expression data is used to construct a statistical model that predicts correctly the “subgroup” of each sample. This training set is then tested with independent data (referred to as a test or validation set) to determine the robustness of the computer-based model. These models are sometimes termed “expert systems,” but may be based on a range of different mathematical procedures such as support vector machine, decision trees, k-nearest neighbour and naïve Bayes. Supervised methods can use a data set with reduced dimensionality (for example, the first few principal components), but typically use unreduced data, with all dimensionality. In all cases the methods allow the quantitative description of the multivariate boundaries that characterise and separate each subtype in terms of its intrinsic gene expression profile. It is also possible to obtain confidence limits on any predictions, for example, a level of probability to be placed on the goodness of fit. The robustness of the predictive models can also be checked using cross-validation, by leaving out selected samples from the analysis.

After stratifying the training samples according to subtype, a centroid-based prediction algorithm may be used to construct centroids based on the expression profile of the gene sets described in Tables 1 and 2.

“Translation” of the descriptor coordinate axes can be useful. Examples of such translation include normalization and mean-centering. “Normalization” may be used to remove sample-to-sample variation. Some commonly used methods for calculating normalization factor include: (i) global normalization that uses all genes on the microarray or nanostring codeset; (ii) housekeeping genes normalization that uses constantly expressed housekeeping/invariant genes; and (iii) internal controls normalization that uses known amount of exogenous control genes added during hybridization (Quackenbush (2002) Nat. Genet. 32 (Suppl.), 496-501). In one embodiment, the genes listed in Tables 1 and 2 can be normalised to one or more control housekeeping genes. Exemplary housekeeping genes include ACTB (60), GAPDH (2597) and TBP (6908), the numbers in brackets following each gene name being the NCBI Gene ID number for that gene; the nucleotide sequence for each gene as disclosed at that NCBI Gene ID number on 18 Jun. 2018 is expressly incorporated herein by reference. It will be understood by one of skill in the art that the methods disclosed herein are not bound by normalization to any particular housekeeping genes, and that any suitable housekeeping gene(s) known in the art can be used. Many normalization approaches are possible, and they can often be applied at any of several points in the analysis. In one embodiment, microarray data is normalised using the LOWESS method, which is a global locally weighted scatterplot smoothing normalization function. In another embodiment, qPCR and NanoString nCounter analysis data is normalised to the geometric mean of set of multiple housekeeping genes. Moreover, qPCR can be analysed using the fold-change method.

“Mean-centering” may also be used to simplify interpretation for data visualisation and computation. Usually, for each descriptor, the average value of that descriptor for all samples is subtracted. In this way, the mean of a descriptor coincides with the origin, and all descriptors are “centered” at zero. In “unit variance scaling,” data can be scaled to equal variance. Usually, the value of each descriptor is scaled by 1/StDev, where StDev is the standard deviation for that descriptor for all samples. “Pareto scaling” is, in some sense, intermediate between mean centering and unit variance scaling. In pareto scaling, the value of each descriptor is scaled by 1/sqrt(StDev), where StDev is the standard deviation for that descriptor for all samples. In this way, each descriptor has a variance numerically equal to its initial standard deviation. The pareto scaling may be performed, for example, on raw data or mean centered data.

“Logarithmic scaling” may be used to assist interpretation when data have a positive skew and/or when data spans a large range, e.g., several orders of magnitude. Usually, for each descriptor, the value is replaced by the logarithm of that value. In “equal range scaling,” each descriptor is divided by the range of that descriptor for all samples. In this way, all descriptors have the same range, that is, 1. However, this method is sensitive to presence of outlier points. In “autoscaling,” each data vector is mean centered and unit variance scaled. This technique is a very useful because each descriptor is then weighted equally, and large and small values are treated with equal emphasis. This can be important for genes expressed at very low, but still detectable, levels.

When comparing data from multiple analyses (e.g., comparing expression profiles for one or more test samples to the centroids constructed from samples collected and analysed in an independent study), it will be necessary to normalise data across these data sets. In one embodiment, Distance Weighted Discrimination (DWD) is used to combine these data sets together (Benito et al. (2004) Bioinformatics 20(1): 105-114, incorporated by reference herein in its entirety). DWD is a multivariate analysis tool that is able to identify systematic biases present in separate data sets and then make a global adjustment to compensate for these biases; in essence, each separate data set is a multi-dimensional cloud of data points, and DWD takes two points clouds and shifts one such that it more optimally overlaps the other.

In some embodiments described herein, the prognostic performance of the gene expression ratio may be assessed utilizing a Cox Proportional Hazards Model Analysis, which is a regression method for survival data that provides an estimate of the hazard ratio and its confidence interval. The Cox model is a well-recognised statistical technique for exploring the relationship between the survival of a patient and particular variables. This statistical method permits estimation of the hazard (i.e., risk) of individuals given their prognostic variables (e.g., gene expression ratio as described herein). The “hazard ratio” is the risk of death at any given time point for patients displaying particular prognostic variables.

Prognosis

An individual grouped with the good prognosis group, may be identified as having a cancer that is sensitive to immunotherapy, e.g. immune checkpoint blockade therapy. They may also be referred to as an individual that responds well to immunotherapy, such as immune checkpoint blockade therapy. An individual grouped with the poor prognosis group, may be identified as having a cancer that is resistant to immunotherapy, such as immune checkpoint blockade therapy.

Where the individual is grouped with the good prognosis group, the individual may be selected for treatment with suitable immunotherapy (e.g. immune checkpoint blockade therapy) as described in further detail below. Where the individual is grouped with the poor prognosis group, the individual may be deselected for treatment with the aforementioned immunotherapy and may, for example, receive surgical treatment, radiotherapy and/or another form of anti-cancer agent (e.g. one or more non-immune chemotherapeutic agents or anti-angiogenic agents).

Whether a prognosis is considered good or poor may vary between cancers and stage of disease. In general terms a good prognosis is one where the overall survival (OS) and/or progression-free survival (PFS) is longer than average for that stage and cancer type. A prognosis may be considered poor if PFS and/or OS is lower than average for that stage and type of cancer. The average may be the median survival OS or PFS.

For example, a prognosis may be considered good if the PFS is >6 months and/or OS >18 months. Similarly, PFS of <6 months or OS of <18 months may be considered poor. In particular PFS of >6 months and/or OS of >18 months may be considered good for advanced cancers.

In general terms, a “good prognosis” is one where survival (OS and/or PFS) of an individual patient can be favourably compared to what is expected in a population of patients within a comparable disease setting. This might be defined as better than median survival (i.e. survival that exceeds that of 50% of patients in population).

“Predicting the response of a cancer patient to a selected treatment” is intended to mean assessing the likelihood that a patient will experience a positive or negative outcome with a particular treatment.

As used herein, “indicative of a positive treatment outcome” refers to an increased likelihood that the patient will experience beneficial results from the selected treatment (e.g. reduction in tumour size, ‘good’ prognostic outcome, improvement in disease-related symptoms and/or quality of life).

“Indicative of a negative treatment outcome” is intended to mean an increased likelihood that the patient will not receive the aforementioned benefits of a positive treatment outcome.

The following is presented by way of example and is not to be construed as a limitation to the scope of the claims.

### EXAMPLES

Materials and Methods

Animals. Wild-type mice used were on a C57BL/6J or Balb/C genetic background (ENVIGO). Rag1^{-/-} and Batf3^{-/-} in a C57BL/6 background were housed and bred at Cancer Research UK Manchester Institute in specific pathogen-free conditions in individually ventilated cages.

Both males and female mice were used in procedures and they were randomly assigned to experimental groups. All procedures involving animals were performed under PPL-70/7701 and PDCC31AAF licenses, in accordance with ARRIVE guidelines and National Home Office regulations under the Animals (Scientific Procedures) Act 1986. Procedures were approved by the Animal Welfare and Ethical Review Bodies (AWERB) of the CRUK Manchester Institute and tumour volumes did not exceed the guidelines set by the Committee of the National Cancer Research Institute (Br J Cancer. 2010 May 25; 102(11):1555-77. doi: 10.1038/sj.bjc.6605642.) as stipulated by the AWERB.

Cancer Cell Lines. Cells were cultured under standard conditions and confirmed to be mycoplasma free. Braf^{V600E} melanoma cell lines were established from C57BL/6 Braf^{+/LSL-V600E};Tyr::CreERT2^{+/o};p16^{INK4a-/-} (Dhomen et al., 2009). CT26, 4T1, and MC38 cells are commercially available. Ptgs2^{-/-} and Ptgs1/Ptgs2^{-/-} cells were generated by CRISPR/Cas9-mediated genome engineering as previously described (Zelenay et al., 2015). To restore COX-2 expression in Ptgs2^{-/-} and Ptgs1/Ptgs2^{-/-} cells, the complete open reading frame of mouse of ptgs2 was cloned from parental Braf^{V600E} melanoma cell line into the retroviral vector pFB. The resulting construct was introduced in Ptgs-deficient cells by standard retroviral transduction. Knockout of Ptgs1, Ptgs2 and regain of COX-2 expression was verified by immunoblotting using anti COX-1 and COX-2 specific antibodies (Cell Signaling) and by monitoring the concentration of PGE₂ in cell supernatants by ELISA (R&D or Cayman chemical).

Mouse Procedures. Tumour cells were harvested by trypsinization, washed three times with PBS, filtered on a 70 μm cell strainer and injected subcutaneously into the flank of recipient mice. Growth profile experiments were performed injecting 1×10⁵ cells in 100 μL of PBS. Tumour tissues analysed at day 4 were harvested from mice injected with 2×10⁶ cells in 100 μL of PBS. Tumour cells were >95% viable at the time of injection as determined by Trypan blue exclusion. Tumour size was quantified as the mean of the longest diameter and its perpendicular and expressed as tumour diameter. For COX-2 inhibition in vivo, celecoxib (LC Laboratories) was administered by oral gavage 30 mg/Kg (in 50% PEG400, 10% DMSO) daily from day 5 after tumour cell injection for 3 weeks. Anti-PD-1 monoclonal antibody (clone RMP1-14, BioXCell) was administered intraperitoneally (i.p.) (200 μg/mouse) from day 5 post-tumour cell inoculation twice weekly for a maximum of six injections. In depletion experiments, mice were treated one day before or from day 7 post-tumour cell injection i.p. with 200 μg of specific Ab (control rat or mouse IgG, anti-Gr1 clone RB6-8C5, anti-NK1.1 clone PK136, anti-ASIALO GM-1, anti-CD4 clone GK1.5 and anti-CD8alpha clone YTS 169.4, all from BioXCell or Biolegend) and then every tree days with 200 μg of the indicated antibody for the entire duration of the experiment. Depletion of neutrophils, NK cells, CD4+ and CD8⁺ T cells was checked by FACS using anti-CD49b-APC (clone DX5), anti-Ly6G-PE-CF594 (clone 1A8), anti CD4-Alexa700 (RM4-5) and anti-CD8alpha-PE (clone 53-6.7) respectively.

Quantitative RT-PCR. Tumours were collected and homogenised and total RNA extracted using RLT lysis buffer (QIAGEN) following the manufacturer's recommendations. RNA was further purified using RNeasy RNA isolation kit (QIAGEN). cDNA was synthesised using 3 μg of total RNA by reverse transcription using High Capacity cDNA archive kit (Applied Biosystems) and quantitative real-time PCR was performed using TaqMan probes (Applied Biosystems) using a QS5 fast real-time PCRsystem (Applied Biosystems) or the Biomark® HD system (FLUIDIGM). Data were analysed with the Δ²CT method (Applied Biosystems, Real-Time PCR Applications Guide).

FACS analysis. For analysis of tumour infiltrating leukocytes, tumours were collected, cut into small pieces and digested with Collagenase IV (200 U/ml) and DNase I (0.2 mg/ml) for 30-40 minutes at 37° C., washed with FACS buffer (PBS containing 2% FCS, 2 mM EDTA and 0.01% sodium azide), filtered on a 70 μm cell strainer and pelleted. The composition of tumour infiltrate was determined by flow cytometry using a combination of the following antibodies: CD45-BV605 (Clone 30-F11); CD11b-BV785 (Clone M1/70); Ly6G-PE-CF594 (Clone 1A8); Ly6C-FITC (Clone AL-21); F4/80-PE-Cy7 (Clone CI: A3-1); anti-MHCII I-A/I-E-Alexa700 (Clone M5/114.15.2), anti-CD11c-PerCP/Cy5.5 (Clone N418), anti-CD103 APC or PE (Clone 2E7) NK1.1-APC or PE (Clone PK136); CD49b-APC (Clone DX5), XCR1-BV421 or Alexa647 (Clone ZET) Siglec-H-BV711 (Clone E50-2440) from BD Bioscience, eBioscience or BioLegend. Fc receptors were saturated with an anti-CD16/32 (clone 2.4G2) 5 minutes before the staining. Cell viability was determined by Aqua LIVE/Dead-405 nm staining (Invitrogen). Live cell counts were calculated from the acquisition of a fixed number of 10 μm latex beads (Coulter) mixed with a known volume of unstained cell suspension. Cells were analysed on a Fortessa X-20 (BD Bioscience).

Confocal Analysis. Tumour tissues were mounted in OCT embedding medium (Thermo Scientific) and stored at −80° C. 8 mm consecutive sections were cut, mounted on Superfrost plus slides (Thermo Scientific) and fixed in 4% paraformaldehyde for 15 min, rehydrated in PBS and blocked in 5% normal goat or donkey (Sigma-Aldrich) serum, 2% BSA in PBS for 2 h at room temperature (RT). Tumour sections were incubated with the following primary antibodies for 2 h at RT or over night at 4° C.: affinity purified anti-Ly6G (Clone 1A8; BD Bioscience) and affinity purified anti-NK1.1-biotynilated (Clone PK136). Sections were then incubated for 1 h at RT with the following species-specific cross-adsorbed detection antibodies: Alexa647-conjugated donkey anti-rat and FITC-conjugated streptavidin from Jackson ImmunoResearch Laboratories and Invitrogen-Molecular Probes, respectively. For DNA detection, DAPI (300 nM; Invitrogen-Molecular Probes) was used. After each step, sections were washed with PBS containing 0.01% (v/v) Tween 20 (VWR Chemicals) and finally mounted with antifade mounting medium FluorPreserve Reagent (Calbiochem) and analysed with an Aperio VERSA 200 scanner (Leica). Negative controls were obtained by omission of the primary antibody. Cell number per high power field (HPF) was calculated using Fiji software version 2.0.0-rc14/1.49.

Bioinformatics Analysis of Patient Cohort Datasets. Clinical and genome-wide mRNA (level 3 RSEM normalised) expression data (Illumina mRNA-seq) from 7811 tumour samples representing 20 cancer types were downloaded from Broad Firehose (http://gdac.broadinstitute.org/runs/stddata_2016_01_28) and cBioportal (http://www.cbioportal.org) on May 2017. To obtain the COX-2 ratio, the ‘cancer-promoting’ and ‘cancer-inhibitory’ inflammatory genes whose expression was regulated by COX-2 activity in the mouse models (FIG. 5A and Zelenay et al. 2015) were computed as follows: PTGS2, VEGFA, CCL2, IL8, CXCL1, CXCL2, CSF3, IL6, IL1B and IL1A were positively correlated (pos) and expressed as:

$$pos = {\sum }_{i = 1}^{{n}_{p}} {G}_{i}^{pos} ( e ) ,$$

CCL5, CXCL9, CXCL10, CXCL11, IL12A, IL12B, IFNG, CD8A, CD8B, GZMA, GZMB, EOMES, PRF1, STAT1 and TBX21 were negatively correlated (neg) and defined as:

$$neg = {\sum }_{i = 1}^{{n}_{n}} {G}_{i}^{n e g} ( e ) ,$$

where nₚ and nₙ are the number of genes in pos and neg groups respectively.

Finally, the COX-2 ratio was calculated as:

$$COX - 2 ratio = \frac{\frac{1}{{n}_{p}} {\sum }_{i = 1}^{{n}_{p}} {G}_{i}^{pos} ( e )}{\frac{1}{{n}_{n}} {\sum }_{i = 1}^{{n}_{n}} {G}_{i}^{neg} ( e )}$$

Kaplan-Meier plots for overall survival were generated at the maximum follow up threshold per each tumour type and the COX-2 ratio was used to segregate high risk from low risk patients. CIBERSORT and xCELL analyses were performed using available online tools (https://cibersort.stanford.edu; http://xcell.ucsf.edu). Quantile normalization was disabled for RNA-seq data analysed by CIBERSORT according to (Jin et al. 2017). In FIG. 5C the neutrophil and the NK cell signatures were defined as the average expression of CSF3R, CXCL1, CXCL2, IL8, CXCR1, CXCR2 (neutrophils) and EOMES, KLRB1, NCR1, NCR3, NKG7, TBX21, CD160, PRF1, GZMA, GZMB, IFNG (NK cells) in LUAD and HNSC datasets. In certain cases, the NK gene cell signature may comprise the genes: KLRF1, CD160, SAMD3, CTSW, NCR1, NCR3 and PTGDR (see FIG. 10E, 10G, 10I and FIG. 11A).

RNA sequencing datasets from (Riaz et al. 2017) and (Mariathasan et al. 2018), were analysed to determine the association of the COX-2 ratio with clinical outcome. Raw counts were downloaded from the gene expression omnibus database (GSE91061) for the melanoma cohort. For the cohort of patients with urothelial carcinoma (Mariathasan et al. 2018) raw counts and source code were obtained by loading the package IMvigor210CoreBiologies into the R statistical computing environment. For consistency across datasets the edgeR package was used to normalise count data and generate count per million reads (CPM Ovalues. A minimum of 0.25 CPM in 10% of the patient population was used as the cut-off for gene inclusion. Transformed CPM values (log 2 (CPM+1)) were then harnessed for downstream analyses. Nanostring data was downloaded from (Chen et al. 2016) and accompanying survival data from (Roh et al. 2017). An additional 8 pre-anti-PD1 samples present in the (Chen et al. 2016) dataset were included in the analysis of the association between COX-2 ratio and response, although survival data was not available for these patients. Log 2-transformed counts were used for further analysis. Once COX-2 ratio values were generated as described above, patient outliers were defined by the ROUT method (Q=1%) calculated on GraphPad Prism. These patients were excluded from further analysis and for comparison with other gene signatures. Patients SAM09c84ec0cf34, SAM85f0a3ac1c45, SAM563d6233dfa2 and SAM7c67b05aa109 were excluded from the urothelial carcinoma cohort and patients 3, 76, 85, 9 and 36 from the Riaz et al., melanoma cohort. In both cohorts, patients with whole transcriptomic data but without response data were not included in survival analyses.

Statistical analysis. For all studies, sample size was defined on the basis of past experience on cancer models, to detect differences of 20% or greater between the groups (10% significance level and 80% power). Values were expressed as mean±SEM or median of biological replicates, as specified.

Unpaired Student's t test, Pearson's correlation, chi-square test, one and two way ANOVA were used as specified. A Mann-Whitney U-test was used in cases of non-Gaussian distribution. Survival curves and hazard ratio were calculated with a Log-rank (Mantel-Cox) test. A ROUT test was applied to exclude outliers. A p value 0.05 (*p<0.05, **p<0.01, ***p<0.001) was considered significant. Statistics were calculated with GraphPad Prism version 7.0a (GraphPad Software).

$$pos = {\sum }_{i = 1}^{{n}_{p}} {G}_{i}^{pos} ( e ) , neg = {\sum }_{i = 1}^{{n}_{n}} {G}_{i}^{neg} ( e ) , COX - 2 ratio = \frac{\frac{1}{{n}_{p}} {\sum }_{i = 1}^{{n}_{p}} {G}_{i}^{pos} ( e )}{\frac{1}{{n}_{n}} {\sum }_{i = 1}^{{n}_{n}} {G}_{i}^{neg} ( e )}$$

### Example 1—Reduced Neutrophil and Increased NK Cell Accumulation in Tumours Formed by COX-Deficient Cells

As previously reported (Zelenay et al., 2015), COX-deficient Braf^{V600E}-driven melanoma cells generated using CRISPR-Cas technology invariably failed to form progressive tumours in immunocompetent mice whereas their COX-competent parental counterpart efficiently evaded immune elimination and grew uncontrolled (FIG. 1A). To definitively demonstrate that these categorical opposing tumour fates resulted from impaired COX activity and exclude the possibility that they were due to unforeseen off-target CRISPR effects, we restored COX-2 expression in COX-1 and COX-2 doubly deficient (Ptgs1^{-/-} Ptgs2^{-/-}) cells by retroviral transduction (FIG. 1A). The COX-2-regained melanoma cells secreted large amounts of PGE₂ in vitro and reacquired the ability to grow progressively in wild-type syngeneic mice as their parental line (FIG. 1A, B).

Having confirmed that the increased immunogenicity of COX-deficient cells could be reverted by restoring COX-2 expression and hence independent of potential off-target CRISPR-mediated introduction of target antigens, we exploited this experimental system to characterise the inflammatory cell composition of the tumour infiltrate. As adaptive immune T cell-mediated control of tumour growth was only apparent 7 to 10 days post-melanoma cell implantation ((Zelenay et al., 2015) and see below), we intentionally chose an earlier time point to pinpoint candidate immune cell subsets responsible for orchestrating the subsequent T cell response. COX-deficient tumours were noticeably smaller already at 4 days and this effect was reversed by COX-2 restoration (FIG. 1C). Examination of various immune cell types by multicolour fluorescence-activated cell sorting (FACS) analysis (FIG. S1A) showed an increase in neutrophils and a marked reduction in NK cell infiltration in tumours formed by parental or COX-2-regained melanoma cells (FIG. 1D, E). Other immune cell populations were unchanged or only moderately and/or inconsistently affected by cancer cell COX-2 competence (FIG. S1B-F). Analysis of tumour sections by immunofluorescence confirmed the above results demonstrating absence of Ly6G⁺ neutrophils and evident clusters of NK1.1+NK cell infiltration in COX-deficient tumours (FIGS. 1F, G)

### Example 2—Lessened Neutrophil and Elevated NK Cell Numbers in COX-Deficient Colorectal and Breast Cancer Models

To assess whether the COX-dependent changes in immune cell composition at the tumour site were unique to the Braf^{V600E}-melanoma model, we extended our analysis to MC38 colon carcinoma cells. These cells expressed COX-2 and produced PGE₂, albeit at significantly lower levels than the melanoma cells (FIG. 2A). Still, CRISPR-mediated ablation of COX-2 totally abrogated PGE₂ production and impaired their ability to form progressive tumours in immunocompetent hosts (FIG. 2A, B). As in the Braf^{V600E} melanoma model, the growth profile of COX-2-deficient (Ptgs2^{-/-}) MC38 cells in T and B-cell-deficient Rag1^{-/-} or cDC1-deficient Batf3^{-/-} mice was comparable to that of parental Ptgs2+/+ or COX-2-restored Ptgs2^{-/-} cells. These observations uncovered a dominant role for cancer cell-intrinsic COX-2 activity in evasion of cDC1- and adaptive immunity-dependent control in this widely studied colon cancer model (FIG. 2B).

Analysis of MC38 tumours early post-implantation also showed conservation with the melanoma model in terms of changes in tumour burden and immune infiltrate composition dependent on COX-2 competence by the cancer cells (FIG. 2C, D). Both neutrophils and NK cells were profoundly affected with a decrease in the former and an increase in the latter in tumours formed by Ptgs2^{-/-} MC38 colorectal cells (FIG. 2D). By contrast, the levels of other myeloid cell subsets, including monocytes, tumour-associated macrophages (TAMs) or DCs were largely unaffected (Supplementary FIG. 2). Reduced numbers of neutrophils but higher accumulation of NK cells, with a trend for lower size and less pronounced or consistent changes in other innate immune cell populations were equally observed in COX-deficient breast 4T1 and CT26 tumours relative to their COX-sufficient counterparts (FIG. 2C, D, Figure S2). Altogether, these data indicate that cancer cell-intrinsic COX-activity favors neutrophil accumulation and hinders NK cell recruitment to tumours across several cancer mouse models independently of cancer type or mouse background.

To investigate the kinetics of leukocyte recruitment into the tumour site we characterised the immune infiltrate at various time points using the Braf^{V600E}-driven melanoma model. Neutrophils and NK cells showed divergent accumulation in COX-competent and deficient tumours already from day 2-post melanoma cell implantation and this difference was maintained for at least one week (FIG. S1G). By 4 weeks, when COX-competent cancer cells have invariably established large and stiff tumours, the proportion of neutrophils and NK cells was comparable to that observed in the few remaining small but still detectable COX-deficient tumours (FIG. S1H). At this late time point, however, Ptgs^{-/-} tumours showed a relative enrichment in cDC1, total CD3⁺ T cells and CD8⁺ T cells in keeping with their immune-dependent remission (FIG. S1H).

As neutrophils were abundant early in COX-competent tumours but not in COX-deficient ones, we next assessed their contribution to tumour burden and immune cell composition depleting them using a monoclonal anti-GR1 antibody. Despite their efficient elimination neither tumour weight nor the prevalence of other leukocyte subset were altered in COX-competent or -deficient tumours (FIGS. S3A and S3B). These results exclude, in turn, a major role for the so-called GR-1⁺ myeloid-derived suppressor cells (MDSCs) in the early innate phase control of COX-deficient melanoma cells.

### Example 3: NK Cells are Essential for Spontaneous or Therapy-Induced Tumour Control

We then addressed the role of NK cells, which have been frequently implicated in the control of hematological malignancies and metastasis but less so of solid tumours (Imai et al., 2000). With the exception of NK cells themselves, the overall immune cell composition of COX-deficient tumours was not evidently altered following NK cell-depletion (FIG. 3A and FIG. S3C). Nonetheless, NK cell-ablation led to a clear increase in Ptgs2^{-/-} tumour size and weight comparable to that of COX-competent tumours, already noticeable at four days post cancer cell implantation (FIG. 3C). Strikingly, moreover, COX-deficient melanoma cells grew progressively as their parental cells in NK cell-depleted mice, with no apparent signs of innate or adaptive immune-dependent control (FIG. 3C). Analogous results were obtained with the MC38 colorectal model (FIG. 3D-F and FIG. S3D). We therefore conclude that NK cells are essential for both the innate and adaptive control phase of COX-deficient tumours.

To further evaluate the relative and hierarchical contribution of the leukocyte subsets involved in tumour eradication, we compared side by side the growth profile of Ptgs^{-/-} Braf^{V600E}-melanoma cells in Rag1^{-/-} mice, Batf3^{-/-} mice missing cDC1 cells or in mice depleted of NK cells, CD4⁺ T cells, CD8⁺ T cells or both CD4⁺ and CD8⁺ T cells.

In agreement with NK cell-involvement in both the innate and adaptive immune phases of tumour control, COX-deficient tumours in NK cell-depleted mice grew faster than in mice singly depleted of CD8⁺ T cells (FIG. 3G). Nonetheless, no tumour regressed in the latter group underscoring a critical role for CTLs, consistent with the progressive tumour growth observed in Rag1^{-/-} or Batf3^{-/-} mice. While tumour regressions still occurred in mice depleted of CD4⁺ T cells, combined ablation of both CD4+ and CD8⁺ cells led to faster growing tumours than in mice ablated of just CD8⁺ T cells underscoring a non-redundant contribution of CD4⁺ T cells to tumour elimination (FIG. 3G). Finally, delaying NK cell-removal for a week did not impair the eradication of COX-deficient tumours revealing a vital and specific role for NK cells early on in the anti-tumour immune response (FIG. 3H). Collectively, our data confirmed cDC1 and CTLs and further identified NK cells as early central players in spontaneous immunity against COX-deficient tumours.

Combinations of PD-1/PD-L1 axis blockade with COX inhibitors have been shown to synergise in promoting immune-mediated tumour regression in preclinical models poorly responsive to either single therapy (Li et al., 2016; Zelenay et al., 2015). To determine whether NK cells were equally required for the therapeutic efficacy of this combination, we treated COX-competent melanoma-bearing mice depleted or not of NK cells with an anti-PD-1 monoclonal antibody and celecoxib, a selective COX-2 inhibitor. The manifest benefit obtained from this treatment, which led to complete responses in more than half of control mice, was totally lost upon depletion of NK cells (FIG. 3 I-K). Tumours grew even faster in anti-PD-1 and celecoxib-treated mice lacking NK cells than in untreated NK cell-sufficient control mice implying residual NK cell activity against COX-competent parental tumours. Together, our results demonstrate a fundamental role for NK cells in natural and therapy-induced control of tumours rendered immunogenic by genetic or therapeutic inhibition of COX-2 activity.

### Example 4: NK Cells Orchestrate a Switch Towards Cancer Restrictive Inflammation

We next sought to determine how NK cells contributed to cDC1 and CTL-dependent tumour control. As their early, but not late depletion, totally abrogated the eradication of Ptgs^{-/-} tumours, we reasoned that NK cells could be involved in the initiation of this process by driving the reprogramming of COX-deficient tumours towards anti-cancer immune pathways. To evaluate this, we measured the expression levels of several inflammatory factors, many of which we previously found to be induced by COX-2 activity (Zelenay et al., 2015) in mice bearing Ptgs^{+/+} or Ptgs^{-/-} tumours, depleted or not of NK cells. Likewise, we monitored the expression of type I immunity-defining markers including mediators of NK cell and CTL recruitment, differentiation and cytotoxic activity. Transcript levels for soluble factors characteristic of cancer-related inflammation, referred as ‘cancer-promoting’, were markedly higher in COX-competent than in COX-deficient tumours and their expression was unchanged or moderately increased in absence of NK cells (FIG. 4A). In contrast, the expression of ‘cancer-inhibitory’ factors comprising hallmarks of cytotoxic immunity was significantly reduced by depletion of NK cells (FIG. 4A, B). This effect was particularly evident in Ptgs^{-/-} tumours in which the expression of these genes was highest in agreement with their spontaneous immune-mediated remissions.

To determine the contribution of NK cells to skewing the local TME relative to that of other immune cell populations required for Ptgs^{-/-} tumour eradication (i.e. T cells and cDC1), we performed parallel analyses in T, B and NKT cell- (Rag1^{-/-}) or cDC1-deficient (Batf3^{-/-}) mice. Notably, the expression of most ‘cancer-inhibitory’ genes was NK cell-dependent but unaltered in Rag1^{-/-} or Batf3^{-/-} hosts indicating a specific, preceding and dominant role for NK cells in orchestrating the inflammatory response of COX-deficient tumours (FIG. 4B). Il12b and Cxcl10 were noteworthy exceptions equally reduced in absence of NK cells or cDC1. The latter findings are in agreement with previous studies indicating cDC1 are a major intratumoural source of IL12 and CXCL10 (Ruffell et al., 2014; Spranger et al., 2017) and that their recruitment to the tumour site is regulated by NK cell activity (Böttcher et al., 2018). Indeed, in line with the latter report, the levels of Xcl1 and Ccl5, as well as those of Ccl3 and Ccl4, also implicated in cDC1 recruitment to the TME (Spranger et al., 2015), were all reduced in tumours from NK cell-depleted mice (FIG. 4B, C). However, the expression of the cDC1-defining markers Clec9a and Xcr1, diminished as expected in Batf3^{-/-} mice, were unaltered by NK cell ablation (FIG. 4C). These findings were consistent with our FACS analysis showing a rather modest contribution of NK cells to intratumoural cDC1 accumulation (FIG. S1B and S2B). We therefore reasoned that NK cells in addition to or rather than attracting cDC1 could alternatively drive their maturation and/or activation locally at the tumour site. To test this, we examined the activation status of tumour-infiltrating cDC1 in NK cell-replete and depleted hosts. The percentage of CD86+ and CD40⁺ cDC1 was significantly higher in COX-deficient tumours than in their parental counterparts as previously reported (Zelenay et al., 2015). Notably, the increase in activated but not total cDC1 was completely abolished following NK cell-depletion (FIG. 4D) indicating that NK cells promote the activation of intratumoural cDC1s. Altogether, our data is consistent with a model whereby NK cells, by orchestrating a switch in the inflammatory profile of Ptgs^{-/-} tumours, initiate a tumour-eradicating response mediated by the sequential action of NK cells, cDC1 and CTLs.

### Example 5: The Mouse COX-2-Driven Signature is Conserved Across Human Malignancies

To investigate if the COX-2 pathway influenced the inflammatory profile of the TME in humans we interrogated large cancer patient datasets. We first analysed the association of COX-2 and the ‘cancer-promoting’ and ‘cancer-inhibitory’ inflammatory mediators whose expression was regulated by COX-2 activity in the mouse models (FIG. 4A). Analysis of 20 different cancer types from The Cancer Genome Atlas (TCGA, https://cancergenome.nih.gov) and METABRIC (Curtis et al., 2012) projects showed unambiguous positive correlations between transcript levels of PTGS2, encoding for COX-2, and factors induced by cancer cell-intrinsic COX-2 in murine tumours across all cancer types (FIG. 5A). Strikingly, moreover, in several of the cancer types analysed PTGS2 showed an inverse correlation with those ‘cancer-inhibitory’ mediators whose expression was elevated in mice bearing COX-deficient tumours (FIG. 5A). These negative correlations were not particularly pronounced, however, individually the vast majority reached statistical significance as shown in lung adenocarcinoma (LUAD) or head and neck squamous cell cancer (HNSC) patient cohorts (FIG. 5B). Additionally, PTGS2 expression positively or negatively correlated with neutrophil- or NK cell-specific gene signatures, respectively (FIG. 5C), suggesting opposed patterns of intratumoural accumulation of neutrophils and NK cells depending on COX-2 levels. These results underscore the translational relevance of our findings in mouse models to human settings and imply that intratumoural COX-2 activity can alter the molecular and cellular infiltrate composition in multiple human malignancies.

### Example 6: A COX-2-Driven Signature Ratio Predicts Overall Patient Survival

To further assess the clinical significance of our findings and evaluate the potential use of a mouse-derived COX-2 signature as a biomarker of patient outcome, we carried out proportional hazard survival analyses in patient datasets with matched clinical outcome data. To integrate both the tumour stimulatory and restrictive elements of the ‘COX-2 signature’ we calculated the ratio between the combined average expression of ‘cancer-promoting’ and ‘cancer-inhibitory’ genes per patient (see methods). Stratification of patients in LUAD, HNSC, triple negative breast cancer (TN_BRCA) and metastatic skin cutaneous melanoma (M_SKCM) according to this ‘COX-2 ratio’ in quartiles showed that those with higher COX-2 ratio had invariably worst outcome (FIG. 6A). In contrast, neither the individual elements of the COX-2 signature nor the combined ‘cancer-promoting’ or ‘cancer-inhibitory’ genes consistently predicted survival across these four tumour types (FIG. 6B). Of note, in the selected datasets, the COX-2 ratio was more consistently, and more powerfully prognostic than CD8⁺ T cell, (Spranger et al., 2015), IFN-γ-related (Ayers et al., 2017) or cDC1 gene signatures (Böttcher et al., 2018) (FIG. 6B), underscoring the value of the ‘COX-2 signature’ and the benefit of integrating pro- and anti-tumourigenic factors in a single biomarker.

### Example 7: The COX-2 Ratio Delineates Tumours with Antagonistic Immune Cell Composition

The inflammatory cell composition of the tumour infiltrate has been associated with both patient overall survival and outcome from treatment (Blank et al., 2016; Fridman et al., 2012; Gentles et al., 2015). Thus, we next evaluated whether the mouse COX-2 signature could be used as a means to discriminate human cancer biopsies with distinct leukocyte infiltrates resorting to recently developed analytical tools to infer the abundance of select immune cell populations (CIBERSORT; https://cibersort.stanford.edu). We focused on immune cell subsets whose intratumoural accumulation was regulated via cancer cell-intrinsic COX-2 and/or required for immune-mediated control in the mouse models. Cancer biopsies with higher COX-2 ratio among LUAD, HNSC, TN_BRCA and M_SKCM cohorts and stratified as in the survival analyses shown in FIG. 6A, had increased relative number of neutrophils and reduced activated NK cells (FIG. 6C). In addition, CD8 T cells and effector memory CD4 T cells were consistently more abundant in biopsies with lower COX-2 ratio. Likewise, in those same samples, the ratio of CD8 T cells to regulatory T cells, a widely used indicator of enhanced anti-tumour T cell activity was markedly elevated (FIG. S6A). Analogous results were obtained using an independent platform (xCell; http://xcell.ucsf.edu) demonstrating enrichment or scarcity in neutrophils, NK cells or CTLs when stratifying samples according to their COX-2 ratio (FIG. S6B).

Among all cell populations whose relative abundance can be inferred with either CIBERSORT (22) or xCell (64) platforms, neither allowed estimating cDC1 infiltration, which were critical for immune-mediated tumour control in our mouse models. To overcome this limitation, we recurred to a published cDC1 gene expression profile obtained by single-cell RNA sequencing (Villani A Science 2017) and performed a custom CIBERSORT analysis. This analysis revealed that cDC1 were significantly more abundant in tumour samples with a lower COX-2 ratio in LUAD and HNSC datasets (FIG. 6D) in agreement with the previously reported association of cDC1 and outcome (Böttcher et al., 2018; Broz et al., 2014; Ruffell et al., 2014). Together, our data provides evidence for marked and broad conservation of COX-2-dependent modulation of the inflammatory profile of the TME across mouse and human species. Furthermore, it implies that our mouse-driven COX-2 ratio constitutes a powerful prognostic biomarker and straightforward means to delineate tumour biopsies with antagonistic inflammatory infiltrates.

### Example 8: The COX-2 Ratio Predicts Outcome from PD-1/PD-L1 Blockade

Finally, to investigate if the COX-2 ratio could be used to predict benefit from ICB, we recurred to available datasets from cancer patients that underwent anti-PD-1 blockade. We analysed three independent publicly available datasets; two of which were melanoma and one bladder cancer patients (Chen et al., 2016; Roh et al., 2017) (Mariathasan et al., 2018; Riaz et al., 2017). In these patient cohorts the expression levels of the individual components of the COX-2 at baseline was available and thus we could calculate the ‘COX-2 ratio’ per patient and determined its association with patient outcome (see methods). In all cases, patient groups that derived benefit from PD-1 blockade, ‘responders’ or ‘non-progressive disease’ as defined in the original studies (Mariathasan et al., 2018; Riaz et al., 2017), the COX-2 ratio was significantly lower than in ‘non-responders’ or ‘progressive disease’ groups (FIG. 7A). Mirroring the overall survival analysis in TCGA and METABRIC datasets, the expression of the individual signature elements was not consistently different between the two groups of patients across the three patient cohorts studies (FIG. S7A). This analysis underlined the value of computing the COX-2 signature as a ratio that integrates cancer-promoting and inhibitory features and suggested its potential use as a biomarker of intrinsic resistance to PD-1 blockade. To further evaluate this possibility we compared the survival outcome following treatment of patients stratified according to their COX-2 ratio at baseline. Patients with a lower COX-2 ratio benefited significantly more than those with a high COX-2 ratio (FIG. 7B) while stratification based on only cancer promoting or inhibitory genes did not or less strongly associated with survival (Supplementary FIG. 7B). The COX-2 predictive power was independent of age and gender (not shown) and notably, once again, it outperformed T cell-, IFN-γ related-, or a cDC1-signature-based stratification (FIG. 7B). In the bladder cancer cohort study, a substantial number of patients experienced full remissions (Mariathasan et al., 2018). Of those complete responders, 90% were among the COX-2 ratio low group further stressing the predictive power of the COX-2 signature. Moreover, the COX-2 ratio mean value showed a gradual decrease within progressive disease, stable disease, partial and complete responder patient groups (FIG. 7C). Equally, survival improved progressively in patients stratified according to the four quartiles (FIG. 7D) indicating a functionally relevant continuum change in the COX-2 signature across all patient subgroups. Finally, the relative cell abundance of activated NK cells but not of CD8 T cells or cDC1, estimated using CIBERSORT, gradually decreased among the four patient clinical subgroups (FIG. 7E and FIG. S7C) mirroring the above results for the COX-2 ratio and supporting a crucial role for NK cells in patient response to ICB. We conclude that the mouse-driven COX-2 ratio constitutes a strong predictive biomarker of response to PD-1/PD-L1 blockade.

Discussion

The dual opposing role of inflammation in cancer is well recognised (Mantovani et al., 2008). Inflammatory signalling and mediators commonly found in clinically apparent tumours enable several features of aggressive tumour growth such as cancer cell proliferation, angiogenesis or invasion (Hanahan and Weinberg, 2011). Others, conversely, have tumour suppressive effects in part by contributing to immune-mediated recognition and killing of cancer cells. The signals that drive or prevent the establishment of tumour microenvironments that support or restrain cancer progression remain poorly understood. Combining the use of cancer mouse models with bioinformatics analysis of patient datasets we here identified the COX-2 pathway as a broadly conserved regulator of the type of intratumoural inflammatory response.

Cancer cell-intrinsic genetic ablation of COX expression impairs the ability of cancer cells to form progressive tumours in immunocompetent but not immunodeficient hosts. The prevalence of this phenomenon is absolute in that immune-mediated tumour growth control is invariably observed regardless of tumour type or strain background. Interestingly, we demonstrate that the radical increase in the immunogenicity of COX-deficient cells is independent of potential antigenic determinants introduced in cancer cells by CRISPR manipulation. Rather, it stemmed from a switch in the immune stimulatory potential of COX-deficient cancer cells evidenced by a profound reprogramming of the intratumoural inflammatory response. This shift took place very early on before any evidence of T cell tumour control and was characterised by reciprocal and antagonistic accumulation of neutrophils and NK cells and by pronounced changes in intratumoural levels of well-established pro- or anti-tumourigenic mediators. Mechanistically, use of mice lacking specific immune subsets by genetic means or through Ab-mediated depletion demonstrated an essential early role for NK cells in the rapid induction of classic anti-cancer immune mediators and in innate and adaptive-immune dependent tumour eradication.

NK cells have been frequently implicated in the control of haematological malignancies but somewhat less so in that of solid tumours (Guillerey et al., 2016). Our findings add to the list of recent studies implying a role for this innate lymphocyte subset in the immunesurveillance of solid neoplasms (Barrow et al., 2018; Lavin et al., 2017; Molgora et al., 2017). Tumour suppressive roles of NK cells are pleiotropic ranging from directly sensing and killing transformed cells to orchestrating and helping CD8 T cell-mediated tumour control (Morvan and Lanier, 2016). In our cancer mouse models, growth control of COX-deficient tumours was as reliant on NK cells as it was as on cDC1 and CTLs. Our parallel growth profile and infiltrate composition analysis of tumour-bearing mice lacking NK cells, cDC1 or CTLs is consistent with a temporal sequence of events whereby NK cells act first instigating the initial anti-tumourigenic inflammatory response, and orchestrating the later CTL-mediated response. Interestingly, NK cells critically contributed to both innate and adaptive phases of tumour immunity whereas Batf3-dependent DCs and T cells, especially the CD8⁺ subset, were uniquely required for the late adaptive immune control. Of note, nonetheless, while rapid NK cell-dependent retardation of tumour growth was evident, it was never sufficient for sustained growth control. The latter, leading to complete full and long-term tumour eradication in the melanoma model, invariably needed NK cells, cross-presenting cDC1 and T cells.

Expression levels of transcripts encoding for cytokine, chemokine and other classic mediators of cytotoxic immunity showed that their induction in tumours was largely dependent on NK cell presence. Notable exceptions were CXCL10 or IL12 whose levels were equally diminished by NK cell or cDC1-deficiency. Both cDC1-derived CXCL10 and IL12 have been recently argued to contribute to the non-redundant role of intratumoural cDC1 in spontaneous and therapy-induced anti-cancer immunity (Mittal et al., 2017; Ruffell et al., 2014; Spranger et al., 2017). Our findings imply thus a crosstalk between NK cells and cDC1 that impacts on the landscape of the TME. Indeed, recent studies uncovered a dominant role for NK cells in attracting cDC1 to the tumour site (Böttcher et al., 2018) by their production of CCL5 and XCL1 (Böttcher et al., 2018) or of FLT3L (Max Krummel Nat Medicine). Accordingly, we found that the intratumoural levels of either CCL5 or XCL1 and of CCL4, also previously implicated in cDC1 recruitment to the TME (Spranger et al., 2015) were markedly reduced upon NK cell ablation. Yet, in contrast, our analysis of the tumour infiltrate composition by different complementary approaches failed to show pronounced or consistent NK cell contribution to intratumoural cDC1 recruitment. We found, instead, that the activation but not the accumulation of cDC1 was entirely conditional to the presence of NK cells. Our observations support a model whereby spontaneous immunity against COX-deficient tumours relies on an NK cell-mediated reprogramming of the inflammatory response that precedes and drives intratumoural cDC1 activation followed by CTL-mediated tumour killing.

This profound immune-dependent suppression of tumour growth was impaired by natural or restored expression of COX-2 in cancer cells. The COX-2/PGE₂ pathway has long been associated with malignant cancer growth (Wang and Dubois, 2010) and our data further shows that it does so in our experimental systems by promoting immune evasion. The specific cellular targets of PGE₂ action remain unknown but given the wide expression of EP2 and EP4 (Furuyashiki and Narumiya, 2011), the PGE₂ receptors that account for the immunomodulatory effects of PGE₂ (Kalinski, 2012), are likely to be several. NK cells, DCs and T cells are all known direct targets of PGE₂ (Kalinski, 2012). Noteworthy, COX-2-driven induction of tumour-promoting factors, such as IL6, IL1β, CXCL1, or CCL2, was no or only modestly affected in hosts lacking NK cells, cDC1 or T and B cells. Our findings are consistent with the notion that targeting tumour-promoting inflammation can be a means to enhance anti-cancer immunity and the efficacy of immunotherapy (Coussens et al., 2013). In particular, we showed that combining anti-PD1 blockade with oral administration of celecoxib, a selective COX-2 inhibitor, strongly hampered cancer growth and promoted complete responses in a large fraction of mice bearing established COX-competent melanomas. This data is in keeping with recent studies, (Hou et al., 2016; Li et al., 2016; Zelenay et al., 2015) and provide further support to the rationale of combining inhibitors of the COX-2 pathway with ICB in the clinic. Of additional direct therapeutical relevance, we show that NK cells were indispensable for treatment benefit suggesting a potential key contribution of this immune cell type to the efficacy of immunotherapy based on ICB.

To evaluate the translational relevance of our findings in cancer mouse models to human cancer, we carried out bioinformatics analysis of large human cancer datasets. In doing so, we found a clear delineation of the type of inflammatory response that correlated, as in the mouse models, with COX-2 expression. This positive and negative association of PTGS2 transcripts with pro- and anti-tumourigenic inflammatory mediators could be found conserved in various cancer types. COX-2 levels positively correlated with tumour-promoting factors irrespectively of cancer type, while the inverse relation with anti-tumour mediators was only observed in selected malignancies. The underlying basis for these observations remains presently unknown. Intriguingly, the anti-correlation of COX-2 transcript levels with the cancer-inhibitory genes was marked for triple negative breast cancer samples but modest or absent for other breast cancer subtypes implying that COX-2 modulatory effect might selectively alter the intratumoural profile within specific cancer subtypes.

The impact of the COX-2 pathway in the intratumoural profile of human cancers also extended to the immune cell composition of the TME. PTGS2 transcript levels significantly associated with higher neutrophil numbers and lower NK cell abundance readily mimicking the findings in the various cancer mouse models. This phenotype, in turn, indicates that COX-2 levels do not simply reflect differential overall leukocyte infiltration but rather qualitative changes in tumour infiltrate cell composition. Inferring the relative abundance of various immune cell populations using two recent and independent available analytical tools additionally supported this conclusion. Moreover, in total agreement with the antagonistic phenotype and fate of murine COX-competent and deficient tumours, T cells, and CTLs in particular, are far more prevalent in biopsies with lower COX-2 ratio. These results suggest that determining the COX-2 ratio could constitute a straightforward way to infer the infiltrate composition of tumours, a readout widely associated with overall survival and more recently with response to ICB (Fridman et al., 2012; Gentles et al., 2015; Mandal and Chan, 2016; Thorsson et al., 2018; Topalian et al., 2016).

COX-2 ratio-based stratification of cancer patient also exposed the remarkable prognostic value of this gene signature whereas neither the individual gene elements nor the combined cancer-promoting or -inhibitory genes showed as strong or consistent prognostic power. We speculate that the superior power of the COX-2 ratio potentially derives from combining surrogate markers of two intimately linked hallmarks of cancer, tumour-promoting inflammation and evasion of immunity destruction (Hanahan and Weinberg, 2011) in one single biomarker. The advantage of multigene gene signatures over single markers is well recognised (Ayers et al., 2017; Broz et al., 2014; Chen et al., 2016) and is of particular value in complex systems as the TME where, arguably, no inflammatory mediator can be attributed exclusive tumour promoting or suppressive properties.

The usefulness of the COX-2 ratio as a predictive biomarker also extended to immunotherapies targeting PD-1. Patients whose tumours at baseline had a lower COX-2 ratio were enriched within the responder group and had significantly improved survival outcome. This was the case across three independent patient cohorts (Mariathasan et al., 2018; Riaz et al., 2017; Roh et al., 2017) and regardless of the method used to determine gene expression levels (Nanostring or RNA sequencing) or tumour type (melanoma or bladder cancer). Notably, we found that the COX-2 signature outperforms in prognostic and predictive power previously published gene signatures. These findings are even more impressive considering the COX-2 signature was entirely derived from the analysis of a handful of inflammatory mediators differentially expressed early on within tumours formed post-implantation of COX-competent or -incompetent murine cancer cells. As such the COX-2 signature has neither been refined nor optimised for human analysis circumventing the issues associated with overfitting and exposing the notable parallels between mice and humans. Efforts to enhance its predictive power and the range of malignancies to which it might display biomarker value are specifically contemplated by the present inventors. Without wishing to be bound by any particular theory, the present inventors consider that an increase in predictability might be achieved by integrating other cancer features known to contribute to the efficacy of immunotherapy such as tumour burden ((Huang et al., 2017) or neoantigen prevalence (McGranahan et al., 2016; Schumacher and Hacohen, 2016; Schumacher and Schreiber, 2015). Especially as the COX-2 signature was obtained from the comparison of murine tumours with radically dissimilar immunogenic potential and fate but arguably identical tumour mutational burden. Preliminary data show that improvement to the COX-2 ratio signature may be achieved by application of bioinformatics regression methods, including elastic net and/or random forest analysis.

In conclusion, the rapid and infallible development of antagonistic inflammatory responses coupled to opposite progressive or regressive tumour fates from our cancer mouse models offered us an ideal experimental system to investigate the signals and principles that regulate the establishment of promoting or inhibitory TME. Together with the in silico validation in large human patient datasets, our analyses demonstrated a key role for NK cells in anti-tumourigenic inflammation and argues for the COX-2/PGE₂ pathway as a major and conserved determinant by which cancer cells modulate their local surrounding environment and avoid immune-mediated elimination. It is particularly notable that the data described herein show that the COX-2 ratio is predictive regardless of the tumour type (e.g. melanoma and bladder) and regardless of the specific monoclonal antibody drug used (e.g. Nivolumab or Atezolizumab). As shown in FIG. 11F, the COX-IS ratio was found to be predictive for outcome following Ipilimubab (anti-CTLA4) treatment of melanoma. Moreover, without wishing to be bound by any particular theory, the present inventors consider that the COX-2 ratio would be predictive of treatment response to other immune checkpoint inhibitors and to non-immune checkpoint blockade immunotherapies.

### Example 9: Inflammatory Score Associated with Cycloxygenase (COX-2 Ratio)

Background

Bladder cancer is a tumour type with high mutational burden, and has seen strong responses to ICB in a subset of patients. Similarly, PD1-targeting antibodies have also been approved in renal cancer (Motzer et al., 2015), more recently in combination with anti-CTLA4 antibodies, as well as drugs that target angiogenesis (Mcdermott et al., 2018, Motzer et al., 2019, Rini et al., 2019). Renal cancers, however, do not have a high TMB compared with other cancer types that have comparable responses to ICB such as lung and bladder cancer (Alexandrov et al., Nat, 2013). Renal carcinomas can be driven by copy number alterations, alterations in the PI3K/AKT/MTOR axis and VHL mutations that lead to an angiogenic switch. TMB itself is a poor indicator of response in renal cancer, but a potentially powerful, predictive biomarker in bladder cancer. Likewise, PDL1 IHC has some utility in bladder cancer, but little biomarker potential in renal cancer as demonstrated in a recent phase 3 study where response rates for Avelumab plus Axitinib were comparable in the PDL1 positive population compared to the whole population (Motzer et al., 2019). From a biomarker perspective there remain open questions in both renal and bladder cancer. (1) In bladder cancer, some reports have suggested that measurement of gene expression signatures can provide meaningful additional predictive power compared to TMB alone (Mariathasan et al., 2018), but it is unclear to what extent this is consistent across different cohorts. (2) In advanced renal cancer, it is unclear as to what the best biomarker for response to ICB is, and how we can select the right treatment for the right patient given the arrival of different combination therapies. Original publications from the two available cohorts (Mariathasan et al., 2018, McDermott et al., 2018) demonstrated firstly that in bladder cancer powerful predictions can be achieved by combining TMB with a T cell effector gene signature and pan-fibroblast TGFβ response signature. In renal cancer it was shown that myeloid inflammation associates with a poor outcome in the single agent Atezolizumab arm, and that T effector signature high, myeloid signature high patients had a poorer PFS compared to T effector high, myeloid low patients.

Methods

All computational analysis was performed using R. First, lowly expressed genes were filtered (CPM <0.25 in 90% cases). Next the edgeR package was utilised to normalise raw counts matrices from the Mariathasan and Mcdermott cohorts. Log 2(CPM+1) values were generated and gene signature scores were calculated by taking the mean expression of each set of genes for each patient. The COX-2 ratio was then calculated by dividing the CP inflammatory signature by the CI inflammatory signature. The caret package was used for all model training. The DMwR package was used in order to utilise the SMOTE method for class balancing.

Mcdermott et al:

Of the gene signatures of interest only the CSF3 gene (part of the CP inflammation signature) was not expressed at sufficient levels to meet cut-off criteria. In total, there were 81 patients treated with single agent Atezolizumab that had response data. First, the entire dataset was modelled, using generalised linear models (GLM) to predict the probability of response using individual signatures or different combinations of gene signatures as the input variables. This set out to ask whether the CP signature added predictive value to the CI signature in this dataset using the chi-squared test to compare nested models. Alone, the CP signature and the COX-2 ratio variables explained a significant level of the variation in patient response, as did NK cells. Interestingly, there was a significant interaction between CP and CI signatures in a multivariate GLM suggesting some interdependency of these two inflammatory scores.

The predictive value of these models was then tested in a more robust manner. To do this a 5-fold cross validation was performed with 100 repeats. These models were trained using Cohen's Kappa as an estimate of model performance. In addition, knowing that there were only 25% (20/81) responders in this cohort, the SMOTE method was used within the model training process to balance the classes. Kappa values were compared from cross-validation between the different models. The model with the highest mean Kappa value was the model combining the CP signature and the T cell-inflamed GEP signature (Ayers et al., 2017). Of note, however, there was no significant difference between this model and the COX-2 ratio alone. Indeed, all the best models combined elements of cancer inhibitory inflammation with the CP inflammation gene signature.

Moreover, a clinical classifier was fitted to determine whether gene signature models predicted outcomes with greater precision than simply utilising routine clinical information. The best predictive models, that contained gene signatures as inputs, outperformed the clinical classifier. A stepwise backwards selection procedure was applied to the clinical classifier and the remaining variables were combined into models with different gene signatures. Subsequently, the best model incorporated the COX-2 ratio with clinical information as well as some genomic markers (liver metastasis, prior nephrectomy, MSKCC classification, CD8A IHC, CD31 IHC, TMB, TNB, PBRM1, SETD2, VHL, MTOR and BAP1 mutation status). In combination with the clinical classifier the COX-2 ratio again achieved robust predictive power similar to other models that combined the CP signature with different signatures representing different aspects of CI inflammation. The mean Cohen's Kappa for the model containing clinical variables, genomic markers and the COX-2 ratio was 0.29, whereas the COX-2 ratio alone had a median Kappa of 0.34, therefore no additional benefit could be gained from incorporating these clinical and genomic parameters. In conclusion, the COX-2 ratio has predictive power in the renal cancer cohort.

Mariathasan et al:

HLA-DQA1 (T cell-inflamed GEP) and CD160 (NK signature) were filtered out due to low expression. The entir cohort (n=298) was modelled to determine whether CP inflammation again added benefit to CI inflammation in order to predict outcomes. Using the chi-squared test to compare nested models the present inventors were able to demonstrate that the CP signature added significant predictive value on top of the CI signature alone. In this instance, there was no added benefit of the interaction term of these two variables. In line with these observations the COX-2 ratio was also able to explain a significant level of variation in patient response, similar to that of the combined CP and CI generalised linear model. Next, the present inventors asked whether CP inflammation added predictive value on top of tumour mutation burden (TMB), which was particularly powerful at predicting outcomes in this dataset. Although, the CP signature did not improve the model containing TMB, the COX-2 ratio was able to add statistically significant predictive power to the model containing only TMB in this dataset (chi-squared P value 0.0367).

Next, cross validation was performed with the exact same methodology as for Mcdermott et al, except 10-fold cross validation rather than 5-fold was performed. This was because the Mariathasan cohort was a larger dataset, such that each fold could still contain sufficient responders. In addition, models were incorporated combining gene signatures with TMB. Of the models tested, the best median Kappa value was the TMB*COX-2 ratio model (0.327). Importantly, the TMB*COX-2 ratio model achieved significantly higher Kappa values in cross validation compared with TMB alone, thereby demonstrating that TMB can be improved by combining it with the COX-2 ratio.

As for the previous dataset, a clinical classifier was constructed utilising a variety of clinical variables that were available in the data. After backwards selection only ECOG score at baseline, and TMB, remained in the model. Next, a model was constructed containing ECOG score, TMB, COX-2 ratio and the interaction term of TMB and COX-2 ratio. A chi-squared test revealed that the COX-2 ratio added predictive value on top of these two variables (p value 0.078, deviance 3.1), and that a further backwards selection failed to remove the COX-2 ratio suggesting again that the COX-2 ratio helps classifying patients into responders and non-responders.

Conclusions:

Overall, several lines of evidence underscore the value of combining measurements of CP inflammation and CI inflammation to aid more precise classification of patients. The COX-2 ratio, a single variable incorporating two different signature scores, was superior to models that combined the individual elements of the ratio itself.

### Example 10: Extension of COX-2 Ratio Analysis to Further Cohorts of Patients Undergoing Immune Checkpoint Blockade (ICB)

The analysis described above in Examples 6-8 has been extended to additional cancer types and additional treatments (see FIGS. 8-11).

Further bioinformatic analysis across different cohorts of patients undergoing immune checkpoint blockade (ICB) shows patient stratification based on their COX-IS within pretreatment biopsies correlates with patient benefit across multiple malignancies (FIGS. 10A and 10B): melanoma, urothelial (bladder), gastric and kidney (renal cell carcinoma).

This analysis also shows that the COX-IS associates with outcome from different immune-checkpoint blockade drugs and combinations in treatment naïve patients or heavily pretreated. Data are shown for monotherapy with anti-PD-L1, anti-PD1 or anti-CTLA4 Abs or combinations of anti-PD-1 and anti-CTLA4 or of anti-PDL-1 and VEGF inhibitors (see FIGS. 11M and 11N).

Additional cohorts of treatment naïve or pretreated patients in melanoma, bladder, renal and gastric cancer patients treated with anti-PDL1, anti-PD-1 or anti-CTLA4 (FIG. 10A) demonstrates that non-responder and responder patients (as defined in each of those studies) have significantly different COX-IS values. Thus, the COX-IS associates with outcome from different immune checkpoint blockade drugs across multiple malignancies.

The COX-inflammatory signature (aka COX-2 ratio, COX-IS or ISAC) is comparable within responders and non-responders within each specific tumour type (FIG. 10B).

The COX-2 signature correlates with outcome (overall survival) in LUAD, HNSC, TNBC, metastatic SKCM (M-SKCM) and CESC (Cervical Squamous Cell Carcinoma and Endocervical Adenocarcinoma) (FIGS. 8A and 8B).

Multivariate analysis demonstrated that the COX-IS is an independent prognostic indicator for overall survival after adjusting for classic clinical parameters (stage, sex, age, and others—dependent on tumour type e.g. HPV+ for HNSC) both in TCGA cohorts (FIG. 9), and in patients treated with ICB (FIG. 11B).

Pan-cancer analysis from TCGA (The Cancer genome atlas) shows that integrating the cancer promoting (CP) and cancer inhibitory (CI) inflammatory mediators identifies high-risk patients (FIGS. 8C and 8D) even when they have high levels of tumour infiltrating CD8⁺ T cells (FIG. 8E e.g. yellow group (second from left) versus blue (fourth from left)).

Data on anti-CTLA-4 (Ipilimumab) treated melanoma patients (data set #2 in FIG. 10 and FIG. 11F from Van Allen et al Science 2015 further underscores the remarkable effect of integrating CP and CI to predict patient outcome. In contrast, stratification based on CI (equivalent to using an IFN-γ signature) does not correlate with outcome.

The additional data demonstrates that the COX-IS adds predictive power to established current biomarkers of treatment response such as tumour mutational burden (TMB) or PDL1 expression (determined by histology in the original papers). It should be noted that while in bladder cancer (dataset #5) the TMB (tumour mutational burden) strongly associates with response, this is not the case for the renal cancer patients (dataset #7). Moreover, as will be appreciated by the skilled person, the sequencing necessary to determine TMB may be onerous or expensive by comparison with the requirements for obtaining the tumour gene expression values that provide the COX-IS ratio. Thus, in some circumstances, the COX-IS signature of the present invention may be quicker, cheaper, more efficient and/or more effective than other biomarkers, such as TMB. COX-IS is herein shown to be predictive across a large variety of cancer types (i.e. pan-cancer) in contrast to the more cancer specific nature of TMB as a predictor (i.e. TMB was predictive in bladder cancer, but not renal cancer).

Comparing clearly-defined groups of responder and non-responders (Progressive disease versus complete responses) in dataset #5, demonstrates that the COX-IS signature is the best predictor of outcome when compared with other previously published gene signatures (including the fibroblast signature reported in the Mariathasan et al Nature study)—see FIG. 11E. By focusing on the clearly-defined groups (progressive diseases versus complete responders) a degree of clinician subjectivity may be removed from subject classification thereby minimising or avoiding misclassifications of outcome status.

### Example 11—Comparison of Alternative Methods for Calculating the COX-2 Ratio

The method for combining CP and CI gene signatures may utilise a mean expression value of the two signatures followed by calculating a ratio of these two values. The present inventors sought to compare this method with other methods of scoring to derive the COX-2 ratio in terms of predictive power and other outputs. Four other methods were tested against the original COX-2 ratio method (Method 1).

For method 2, signatures were scored by calculating the mean Z-score for CP and CI signatures, before subtracting CI from CP.

For Method 3, the median value for each gene was calculated across the entire cohort (Mariathasan n=348, Mcdermott n=263). For each gene, a value of +1 was applied if the expression value was greater than the median of that gene over the population. The sum of CI gene scores was then subtracted from the sum of CP gene score, after it was normalised by gene length—that is multiplied by the length of CI divided by the length of CP.

Methods 4 and 5 used similar approaches to Method 3 except Z-scores were utilised, and rather than a median cut-off, a three part scoring system was used. A Z-score >0.1 allocated a +1 score, and <−0.1 a −1 score. Between the 0.1 and −0.1 cut-offs a score of 0 was applied. Again, the sum of CI scores was subtracted from the sum of CP scores. For Method 5, the cut-off used was instead 0.3/−0.3.

In the urothelial carcinoma cohort, the COX-2 ratio (Method 1) had the most significant difference between responders and non-responders (FIG. 12, top). In renal cancer (FIG. 12, bottom), Method 3 most powerfully separated responders and non-responders. Yet, similar to the Mariathasan cohort, there was not a substantial degree of difference between the scoring methods indicating different methods for determining the COX-2 ratio can be equally informative. The pertinent point is that combining the CP inflammation signature with the CI inflammation signature has predictive value that is greater than using either signature alone, and that this approach can seemingly provide additional prognostic and predictive information on top of clinical features such as sex, age, race and metastasis. In addition, there is evidence that this approach could improve upon the predictive power of genomic and transcriptomic biomarkers that have been used clinically such as tumour mutational burden (TMB) and PD-L1 immunohistochemistry. Whether using a Z-score based method or utilising the expression values themselves to generate a score, the combination of these two gene sets is what appears to be most important.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

### REFERENCES

Ayers, M., Lunceford, J., Nebozhyn, M., Murphy, E., Loboda, A., Kaufman, D. R., Albright, A., Cheng, J. D., Kang, S. P., Shankaran, V., Piha-Paul, S. A., Yearley, J., Seiwert, T. Y., Ribas, A., McClanahan, T. K., 2017. IFN-γ-related mRNA profile predicts clinical response to PD-1 blockade. J. Clin. Invest. 127. doi:10.1172/JC191190
Barrow, A. D., Edeling, M. A., Trifonov, V., Luo, J., Goyal, P., Bohl, B., Bando, J. K., Kim, A. H., Walker, J., Andahazy, M., Bugatti, M., Melocchi, L., Vermi, W., Fremont, D. H., Cox, S., Cella, M., Schmedt, C., Colonna, M., 2018. Natural Killer Cells Control Tumor Growth by Sensing a Growth Factor. Cell 172, 534-548.e19. doi:10.1016/j.cell.2017.11.037
Binnewies, M., Roberts, E. W., Kersten, K., Chan, V., Fearon, D. F., Merad, M., Coussens, L. M., Gabrilovich, D. I., Ostrand-Rosenberg, S., Hedrick, C. C., Vonderheide, R. H., Pittet, M. J., Jain, R. K., Zou, W., Howcroft, T. K., Woodhouse, E. C., Weinberg, R. A., Krummel, M. F., 2018. Understanding the tumor immune microenvironment (TIME) for effective therapy. Nature Medicine 24, 541-550. doi:10.1038/s41591-018-0014-x
Blank, C. U., Haanen, J. B., Ribas, A., Schumacher, T. N., 2016. CANCER IMMUNOLOGY. The “cancer immunogram”. Science 352, 658-660. doi:10.1126/science.aaf2834
Böttcher, J. P., Bonavita, E., Chakravarty, P., Blees, H., Cabeza-Cabrerizo, M., Sammicheli, S., Rogers, N. C., Sahai, E., Zelenay, S., Reis e Sousa, C., 2018. NK Cells Stimulate Recruitment of cDC1 into the Tumor Microenvironment Promoting Cancer Immune Control. Cell 172, 1022-1037.e14. doi:10.1016/j.cell.2018.01.004
Broz, M. L., Binnewies, M., Boldajipour, B., Nelson, A. E., Pollack, J. L., Erle, D. J., Barczak, A., Rosenblum, M. D., Daud, A., Barber, D. L., Amigorena, S., Van′t Veer, L. J., Sperling, A. I., Wolf, D. M., Krummel, M. F., 2014. Dissecting the tumor myeloid compartment reveals rare activating antigen-presenting cells critical for T cell immunity. Cancer Cell 26, 638-652. doi:10.1016/j.ccell.2014.09.007
Chen, P.-L., Roh, W., Reuben, A., Cooper, Z. A., Spencer, C. N., Prieto, P. A., Miller, J. P., Bassett, R. L., Gopalakrishnan, V., Wani, K., De Macedo, M. P., Austin-Breneman, J. L., Jiang, H., Chang, Q., Reddy, S. M., Chen, W.-S., Tetzlaff, M. T., Broaddus, R. J., Davies, M. A., Gershenwald, J. E., Haydu, L., Lazar, A. J., Patel, S. P., Hwu, P., Hwu, W.-J., Diab, A., Glitza, I. C., Woodman, S. E., Vence, L. M., Wistuba, I. I., Amaria, R. N., Kwong, L. N., Prieto, V., Davis, R. E., Ma, W., Overwijk, W. W., Sharpe, A. H., Hu, J., Futreal, P. A., Blando, J., Sharma, P., Allison, J. P., Chin, L., Wargo, J. A., 2016. Analysis of Immune Signatures in Longitudinal Tumor Samples Yields Insight into Biomarkers of Response and Mechanisms of Resistance to Immune Checkpoint Blockade. Cancer Discovery 6, 827-837. doi:10.1158/2159-8290.CD-15-1545
Coussens, L. M., Zitvogel, L., Palucka, A. K., 2013. Neutralizing tumor-promoting chronic inflammation: a magic bullet? Science 339, 286-291. doi:10.1126/science.1232227
Curtis, C., Shah, S. P., Chin, S.-F., Turashvili, G., Rueda, O. M., Dunning, M. J., Speed, D., Lynch, A. G., Samarajiwa, S., Yuan, Y., Grdf, S., Ha, G., Haffari, G., Bashashati, A., Russell, R., McKinney, S., METABRIC Group, Langerød, A., Green, A., Provenzano, E., Wishart, G., Pinder, S., Watson, P., Markowetz, F., Murphy, L., Ellis, I., Purushotham, A., Børresen-Dale, A.-L., Brenton, J. D., Tavaré, S., Caldas, C., Aparicio, S., 2012. The genomic and transcriptomic architecture of 2,000 breast tumours reveals novel subgroups. Nature 486, 346-352. doi:10.1038/nature10983
De Henau, O., Rausch, M., Winkler, D., Campesato, L. F., Liu, C., Cymerman, D. H., Budhu, S., Ghosh, A., Pink, M., Tchaicha, J., Douglas, M., Tibbitts, T., Sharma, S., Proctor, J., Kosmider, N., White, K., Stern, H., Soglia, J., Adams, J., Palombella, V. J., McGovern, K., Kutok, J. L., Wolchok, J. D., Merghoub, T., 2016. Overcoming resistance to checkpoint blockade therapy by targeting PI3Kγ in myeloid cells. Nature 539, 443-447. doi:10.1038/nature20554
Dhomen, N., Reis-Filho, J. S., da Rocha Dias, S., Hayward, R., Savage, K., Delmas, V., Larue, L., Pritchard, C., Marais, R., 2009. Oncogenic Braf Induces Melanocyte Senescence and Melanoma in Mice. Cancer Cell 15, 294-303. doi:10.1016/j.ccr.2009.02.022
Fridman, W.-H., Pages, F., Sautes-Fridman, C., Galon, J., 2012. The immune contexture in human tumours: impact on clinical outcome. Nature Reviews Cancer 12, 298-306. doi:10.1038/nrc3245
Furuyashiki, T., Narumiya, S., 2011. Stress responses: the contribution of prostaglandin E(2) and its receptors. Nat Rev Endocrinol 7, 163-175. doi:10.1038/nrendo.2010.194
Gajewski, T. F., Schreiber, H., Fu, Y.-X., 2013. Innate and adaptive immune cells in the tumor microenvironment. Nat. Immunol. 14, 1014-1022. doi:10.1038/ni.2703
Galon, J., 2006. Type, Density, and Location of Immune Cells Within Human Colorectal Tumors Predict Clinical Outcome. Science 313, 1960-1964. doi:10.1126/science.1129139
Gentles, A. J., Newman, A. M., Liu, C. L., Bratman, S. V., Feng, W., Kim, D., Nair, V. S., Xu, Y., Khuong, A., Hoang, C. D., Diehn, M., West, R. B., Plevritis, S. K., Alizadeh, A. A., 2015. The prognostic landscape of genes and infiltrating immune cells across human cancers. Nature Medicine 21, 938-945. doi:10.1038/nm.3909
Guillerey, C., Huntington, N. D., Smyth, M. J., 2016. Targeting natural killer cells in cancer immunotherapy. Nat. Immunol. 17, 1025-1036. doi:10.1038/ni.3518
Hanahan, D., Weinberg, R. A., 2011. Hallmarks of Cancer: The Next Generation. Cell 144, 646-674. doi:10.1016/j.cell.2011.02.013
Herbst, R. S., Soria, J.-C., Kowanetz, M., Fine, G. D., Hamid, O., Gordon, M. S., Sosman, J. A., McDermott, D. F., Powderly, J. D., Gettinger, S. N., Kohrt, H. E. K., Horn, L., Lawrence, D. P., Rost, S., Leabman, M., Xiao, Y., Mokatrin, A., Koeppen, H., Hegde, P. S., Mellman, I., Chen, D. S., Hodi, F. S., 2014. Predictive correlates of response to the anti-PD-L1 antibody MPDL3280A in cancer patients. Nature 515, 563-567. doi:10.1038/nature14011
Hou, W., Sampath, P., Rojas, J. J., Thorne, S. H., 2016. Oncolytic Virus-Mediated Targeting of PGE2 in the Tumor Alters the Immune Status and Sensitizes Established and Resistant Tumors to Immunotherapy. Cancer Cell 30, 108-119. doi:10.1016/j.ccell.2016.05.012
Huang, A. C., Postow, M. A., Orlowski, R. J., Mick, R., Bengsch, B., Manne, S., Xu, W., Harmon, S., Giles, J. R., Wenz, B., Adamow, M., Kuk, D., Panageas, K. S., Carrera, C., Wong, P., Quagliarello, F., Wubbenhorst, B., D'Andrea, K., Pauken, K. E., Herati, R. S., Staupe, R. P., Schenkel, J. M., McGettigan, S., Kothari, S., George, S. M., Vonderheide, R. H., Amaravadi, R. K., Karakousis, G. C., Schuchter, L. M., Xu, X., Nathanson, K. L., Wolchok, J. D., Gangadhar, T. C., Wherry, E. J., 2017. T-cell invigoration to tumour burden ratio associated with anti-PD-1 response. Nature 545, 60-65. doi:10.1038/nature22079
Imai, K., Matsuyama, S., Miyake, S., Suga, K., Nakachi, K., 2000. Natural cytotoxic activity of peripheral-blood lymphocytes and cancer incidence: an 11-year follow-up study of a general population. Lancet 356, 1795-1799. doi:10.1016/S0140-6736(00)03231-1
Kalinski, P., 2012. Regulation of immune responses by prostaglandin E2. J. Immunol. 188, 21-28. doi:10.4049/jimmunol.1101029
Lavin, Y., Kobayashi, S., Leader, A., Amir, E.-A. D., Elefant, N., Bigenwald, C., Remark, R., Sweeney, R., Becker, C. D., Levine, J. H., Meinhof, K., Chow, A., Kim-Shulze, S., Wolf, A., Medaglia, C., Li, H., Rytlewski, J. A., Emerson, R. O., Solovyov, A., Greenbaum, B. D., Sanders, C., Vignali, M., Beasley, M. B., Flores, R., Gnjatic, S., Pe'er, D., Rahman, A., Amit, I., Merad, M., 2017. Innate Immune Landscape in Early Lung Adenocarcinoma by Paired Single-Cell Analyses. Cell 169, 750-765.e17. doi:10.1016/j.cell.2017.04.014
Li, Y., Fang, M., Zhang, J., Wang, J., Song, Y., Shi, J., Li, W., Wu, G., Ren, J., Wang, Z., Zou, W., Wang, L., 2016. Hydrogel dual delivered celecoxib and anti-PD-1 synergistically improve antitumor immunity. OncoImmunology 5, e1074374. doi:10.1080/2162402X.2015.1074374
Mandal, R., Chan, T. A., 2016. Personalized Oncology Meets Immunology: The Path toward Precision Immunotherapy. Cancer Discovery 6, 703-713. doi:10.1158/2159-8290.CD-16-0146
Mantovani, A., Allavena, P., Sica, A., Balkwill, F., 2008. Cancer-related inflammation. Nature 454, 436-444. doi:10.1038/nature07205
Mariathasan, S., Turley, S. J., Nickles, D., Castiglioni, A., Yuen, K., Wang, Y., Kadel, E. E., Koeppen, H., Astarita, J. L., Cubas, R., Jhunjhunwala, S., Banchereau, R., Yang, Y., Guan, Y., Chalouni, C., Ziai, J., Şenbabao\check{g}lu, Y., Santoro, S., Sheinson, D., Hung, J., Giltnane, J. M., Pierce, A. A., Mesh, K., Lianoglou, S., Riegler, J., Carano, R. A. D., Eriksson, P., Höglund, M., Somarriba, L., Halligan, D. L., van der Heijden, M. S., Loriot, Y., Rosenberg, J. E., Fong, L., Mellman, I., Chen, D. S., Green, M., Derleth, C., Fine, G. D., Hegde, P. S., Bourgon, R., Powles, T., 2018. TGFβ attenuates tumour response to PD-L1 blockade by contributing to exclusion of T cells. Nature 554, 544-548. doi:10.1038/nature25501
McGranahan, N., Furness, A. J. S., Rosenthal, R., Ramskov, S., Lyngaa, R., Saini, S. K., Jamal-Hanjani, M., Wilson, G. A., Birkbak, N. J., Hiley, C. T., Watkins, T. B. K., Shafi, S., Murugaesu, N., Mitter, R., Akarca, A. U., Linares, J., Marafioti, T., Henry, J. Y., Van Allen, E. M., Miao, D., Schilling, B., Schadendorf, D., Garraway, L. A., Makarov, V., Rizvi, N. A., Snyder, A., Hellmann, M. D., Merghoub, T., Wolchok, J. D., Shukla, S. A., Wu, C. J., Peggs, K. S., Chan, T. A., Hadrup, S. R., Quezada, S. A., Swanton, C., 2016. Clonal neoantigens elicit T cell immunoreactivity and sensitivity to immune checkpoint blockade. Science aaf1490. doi:10.1126/science.aaf1490
Mittal, D., Vijayan, D., Putz, E. M., Aguilera, A. R., Markey, K. A., Straube, J., Kazakoff, S., Nutt, S. L., Takeda, K., Hill, G. R., Waddell, N., Smyth, M. J., 2017. Interleukin-12 from CD103+ Batf3-Dependent Dendritic Cells Required for NK-Cell Suppression of Metastasis. Cancer Immunol Res 5, 1098-1108. doi:10.1158/2326-6066.CIR-17-0341
Molgora, M., Bonavita, E., Ponzetta, A., Riva, F., Barbagallo, M., Jaillon, S., Popović, B., Bernardini, G., Magrini, E., Gianni, F., Zelenay, S., Jonjić, S., Santoni, A., Garlanda, C., Mantovani, A., 2017. IL-1R8 is a checkpoint in NK cells regulating anti-tumour and anti-viral activity. Nature 551, 110-114. doi:10.1038/nature24293
Morvan, M. G., Lanier, L. L., 2016. NK cells and cancer: you can teach innate cells new tricks. Nature Reviews Cancer 16, 7-19. doi:10.1038/nrc.2015.5
Riaz, N., Havel, J. J., Makarov, V., Desrichard, A., Urba, W. J., Sims, J. S., Hodi, F. S., Martin-Algarra, S., Mandal, R., Sharfman, W. H., Bhatia, S., Hwu, W.-J., Gajewski, T. F., Slingluff, C. L., Chowell, D., Kendall, S. M., Chang, H., Shah, R., Kuo, F., Morris, L. G. T., Sidhom, J.-W., Schneck, J. P., Horak, C. E., Weinhold, N., Chan, T. A., 2017. Tumor and Microenvironment Evolution during Immunotherapy with Nivolumab. Cell 171, 934-949.e15. doi:10.1016/j.cell.2017.09.028
Ribas, A., Wolchok, J. D., 2018. Cancer immunotherapy using checkpoint blockade. Science 359, 1350-1355. doi:10.1126/science.aar4060
Roh, W., Chen, P.-L., Reuben, A., Spencer, C. N., Prieto, P. A., Miller, J. P., Gopalakrishnan, V., Wang, F., Cooper, Z. A., Reddy, S. M., Gumbs, C., Little, L., Chang, Q., Chen, W.-S., Wani, K., De Macedo, M. P., Chen, E., Austin-Breneman, J. L., Jiang, H., Roszik, J., Tetzlaff, M. T., Davies, M. A., Gershenwald, J. E., Tawbi, H., Lazar, A. J., Hwu, P., Hwu, W.-J., Diab, A., Glitza, I. C., Patel, S. P., Woodman, S. E., Amaria, R. N., Prieto, V. G., Hu, J., Sharma, P., Allison, J. P., Chin, L., Zhang, J., Wargo, J. A., Futreal, P. A., 2017. Integrated molecular analysis of tumor biopsies on sequential CTLA-4 and PD-1 blockade reveals markers of response and resistance.
Science Translational Medicine 9, eaah3560. doi:10.1126/scitranslmed.aah3560
Rooney, M. S., Shukla, S. A., Wu, C. J., Getz, G., Hacohen, N., 2015. Molecular and genetic properties of tumors associated with local immune cytolytic activity. Cell 160, 48-61. doi:10.1016/j.cell.2014.12.033
Ruffell, B., Chang-Strachan, D., Chan, V., Rosenbusch, A., Ho, C. M. T., Pryer, N., Daniel, D., Hwang, E. S., Rugo, H. S., Coussens, L. M., 2014. Macrophage IL-10 blocks CD8⁺ T cell-dependent responses to chemotherapy by suppressing IL-12 expression in intratumoral dendritic cells. Cancer Cell 26, 623-637. doi:10.1016/j.ccell.2014.09.006
SAnchez-Paulete, A. R., Cueto, F. J., Martinez-López, M., Labiano, S., Morales-Kastresana, A., Rodriguez-Ruiz, M. E., Jure-Kunkel, M., Azpilikueta, A., Aznar, M. A., Quetglas, J. I., Sancho, D., Melero, I., 2016. Cancer Immunotherapy with Immunomodulatory Anti-CD137 and Anti-PD-1 Monoclonal Antibodies Requires BATF3-Dependent Dendritic Cells. Cancer Discovery 6, 71-79. doi:10.1158/2159-8290.CD-15-0510
Schumacher, T. N., Hacohen, N., 2016. Neoantigens encoded in the cancer genome. Current Opinion in Immunology 41, 98-103. doi:10.1016/j.coi.2016.07.005
Schumacher, T. N., Schreiber, R. D., 2015. Neoantigens in cancer immunotherapy. Science 348, 69-74. doi:10.1126/science.aaa4971
Spranger, S., Bao, R., Gajewski, T. F., 2015. Melanoma-intrinsic R-catenin signalling prevents anti-tumour immunity. Nature 523, 231-235. doi:10.1038/nature14404
Spranger, S., Dai, D., Horton, B., Gajewski, T. F., 2017. Tumor-Residing Batf3 Dendritic Cells Are Required for Effector T Cell Trafficking and Adoptive T Cell Therapy. Cancer Cell 31, 711-723.e4. doi:10.1016/j.ccell.2017.04.003
Spranger, S., Gajewski, T. F., 2018. Impact of oncogenic pathways on evasion of antitumour immune responses. Nature Reviews Cancer 18, 139-147. doi:10.1038/nrc.2017.117
Thorsson, V., Gibbs, D. L., Brown, S. D., Wolf, D., Bortone, D. S., Ou Yang, T.-H., Porta-Pardo, E., Gao, G. F., Plaisier, C. L., Eddy, J. A., Ziv, E., Culhane, A. C., Paull, E. O., Sivakumar, I. K. A., Gentles, A. J., Malhotra, R., Farshidfar, F., Colaprico, A., Parker, J. S., Mose, L. E., Vo, N. S., Liu, J., Liu, Y., Rader, J., Dhankani, V., Reynolds, S. M., Bowlby, R., Califano, A., Cherniack, A. D., Anastassiou, D., Bedognetti, D., Rao, A., Chen, K., Krasnitz, A., Hu, H., Malta, T. M., Noushmehr, H., Pedamallu, C. S., Bullman, S., Ojesina, A. I., Lamb, A., Zhou, W., Shen, H., Choueiri, T. K., Weinstein, J. N., Guinney, J., Saltz, J., Holt, R. A., Rabkin, C. E., Cancer Genome Atlas Research Network, Lazar, A. J., Serody, J. S., Demicco, E. G., Disis, M. L., Vincent, B. G., Shmulevich, L., 2018. The Immune Landscape of Cancer. Immunity 48, 812-830.e14. doi:10.1016/j.immuni.2018.03.023
Topalian, S. L., Taube, J. M., Anders, R. A., Pardoll, D. M., 2016. Mechanism-driven biomarkers to guide immune checkpoint blockade in cancer therapy. Nature Reviews Cancer 16, 275-287. doi:10.1038/nrc.2016.36
Tumeh, P. C., Harview, C. L., Yearley, J. H., Shintaku, I. P., Taylor, E. J. M., Robert, L., Chmielowski, B., Spasic, M., Henry, G., Ciobanu, V., West, A. N., Carmona, M., Kivork, C., Seja, E., Cherry, G., Gutierrez, A. J., Grogan, T. R., Mateus, C., Tomasic, G., Glaspy, J. A., Emerson, R. O., Robins, H., Pierce, R. H., Elashoff, D. A., Robert, C., Ribas, A., 2014. PD-1 blockade induces responses by inhibiting adaptive immune resistance. Nature 515, 568-571. doi:10.1038/nature13954
Vesely, M. D., Kershaw, M. H., Schreiber, R. D., Smyth, M. J., 2011. Natural Innate and Adaptive Immunity to Cancer. Annu. Rev. Immunol. 29, 235-271. doi:10.1146/annurev-immunol-031210-101324
Wang, D., Dubois, R. N., 2010. Eicosanoids and cancer. Nature Reviews Cancer 10, 181-193. doi:10.1038/nrc2809
Zelenay, S., van der Veen, A. G., Böttcher, J. P., Snelgrove, K. J., Rogers, N., Acton, S. E., Chakravarty, P., Girotti, M. R., Marais, R., Quezada, S. A., Sahai, E., Reis e Sousa, C., 2015. Cyclooxygenase-Dependent Tumor Growth through Evasion of Immunity. Cell 162, 1257-1270. doi:10.1016/j.cell.2015.08.015

1. A method for predicting the treatment response to anti-cancer immunotherapy of a mammalian cancer patient, the method comprising:
a) measuring the gene expression of at least 2, 3, 4, 5, 6, 7, 8, 9 or more (such as all of) the following cancer promoting genes: PTGS2, VEGFA, CCL2, IL8, CXCL2, CXCL1, CSF3, IL6, IL1B and IL1A in a sample obtained from the tumour of the patient;
b) measuring the gene expression of at least 2, 3, 4, 5, 6, 7, 8, 9, 10, 11, 12, 13, 14 or more (such as all of) the following cancer inhibitory genes: CXCL11, CXCL10, CXCL9, CCL5, TBX21, EOMES, CD8B, CD8A, PRF1, GZMB, GZMA, STAT1, IFNG, IL12B and IL12A in a sample obtained from the tumour of the patient;
c) computing a ratio of the gene expression of said at least 2 cancer promoting genes and the gene expression of said at least 2 cancer inhibitory genes; and
d) making a prediction of the treatment response and/or prognosis of the patient based on the gene expression ratio computed in step c).

2. The method of claim 1, wherein said ratio is of the gene expression of all said cancer promoting genes PTGS2, VEGFA, CCL2, IL8, CXCL2, CXCL1, CSF3, IL6, IL1B and IL1A and all of said cancer inhibitory genes CXCL11, CXCL10, CXCL9, CCL5, TBX21, EOMES, CD8B, CD8A, PRF1, GZMB, GZMA, STAT1, IFNG, IL12B and IL12A.

3. The method of claim 1 or claim 2, wherein said ratio is calculated according to the formula:
$$COX - 2 ratio = \frac{\frac{1}{{n}_{p}} {\sum }_{i = 1}^{{n}_{p}} {G}_{i}^{pos} ( e )}{\frac{1}{{n}_{n}} {\sum }_{i = 1}^{{n}_{n}} {G}_{i}^{neg} ( e )}$$
wherein nₚ is the number of said cancer promoting genes and nₙ is the number of said cancer inhibitory genes, Gᵢ^{pos} and Gᵢ^{neg} are the positive and negative correlated genes, respectively, within an (i) interval of unitary values, (e) represents the gene expression values, expressed as log 2 counts per million (CPM).

4. The method of any one of the preceding claims, wherein the expression level of each of said genes is a normalised gene expression level.

5. The method of any one of the preceding claims, wherein the gene expression ratio computed in step c) is referenced to the median gene expression ratio of a sample cohort of cancer patients having the same type of cancer as said cancer patient, which median gene expression ratio serves as a threshold, and wherein:
a computed gene expression ratio above said threshold indicates that said cancer patient is at high risk of a poor treatment response to said anti-cancer immunotherapy and/or at high risk of having a shorter survival time than the median survival time of said sample cohort of cancer patients; and
a computed gene expression ratio below said threshold indicates that said cancer patient is at low risk of a poor treatment response to said anti-cancer immunotherapy and/or at low risk of having a shorter survival time than the median survival time of said sample cohort of cancer patients.

6. The method of claim 1 or claim 2, wherein said ratio is calculated by:
computing the mean gene expression Z-score for said at least 2 cancer promoting genes and the mean gene expression Z-score for said at least 2 cancer inhibitory genes, wherein said z-score is calculated according to the formula
$$z = \frac{x - μ}{σ}$$
wherein z is the gene expression z-score of a given gene, x is the gene expression of the given gene, μ is the mean expression of the given gene in a training set comprising a plurality of cancer subjects and σ is the standard deviation of the gene expression of the given gene in the training set; and
subtracting the Z-score for said at least 2 cancer inhibitory genes from the Z-score for said at least 2 cancer promoting genes.

7. The method of claim 1 or claim 2, wherein said ratio is calculated by:
computing the median gene expression value for each of said at least two cancer promoting genes and said at least two cancer inhibitory genes across a training set comprising a plurality of cancer subjects,
applying, for each of said genes, a value of +1 where the expression value of said cancer patient is greater than the median of that gene over the training set,
summing the cancer inhibitory gene scores and summing the cancer promoting gene scores, and
subtracting the summed cancer inhibitory gene score from the summed cancer promoting gene score, optionally after normalising in order to account for the number of cancer inhibitory genes and the number of cancer promoting genes, respectively.

8. The method of claim 1 or claim 2, wherein said ratio is calculated by:
computing the mean gene expression Z-score for said at least 2 cancer promoting genes and the mean gene expression Z-score for said at least 2 cancer inhibitory genes wherein said z-score is calculated according to the formula
$$z = \frac{x - μ}{σ}$$
wherein z is the gene expression z-score of a given gene, x is the gene expression of the given gene, μ is the mean expression of the given gene in a training set comprising a plurality of cancer subjects and σ is the standard deviation of the gene expression of the given gene in the training set;
applying, for each of said genes, a value of +1 where the z-score is greater than 0.1, a value of −1 where the z-score is less than −0.1, and a value of 0 where the z-score is between 0.1 and −0.1;
summing the cancer inhibitory gene applied values and summing the cancer promoting gene applied values, and
subtracting the summed cancer inhibitory gene applied values from the summed cancer promoting gene applied values.

9. The method of claim 1 or claim 2, wherein said ratio is calculated by:
computing the mean gene expression Z-score for said at least 2 cancer promoting genes and the mean gene expression Z-score for said at least 2 cancer inhibitory genes wherein said z-score is calculated according to the formula
$$z = \frac{x - μ}{σ}$$
wherein z is the gene expression z-score of a given gene, x is the gene expression of the given gene, μ is the mean expression of the given gene in a training set comprising a plurality of cancer subjects and σ is the standard deviation of the gene expression of the given gene in the training set;
applying, for each of said genes, a value of +1 where the z-score is greater than 0.3, a value of −1 where the z-score is less than −0.3, and a value of 0 where the z-score is between 0.3 and −0.3;
summing the cancer inhibitory gene applied values and summing the cancer promoting gene applied values, and
subtracting the summed cancer inhibitory gene applied values from the summed cancer promoting gene applied values.

10. The method of any one of the preceding claims, wherein the method further comprises assessing the tumour burden and/or neoantigen prevalence of the cancer patient.

11. The method of any one of the preceding claims, wherein the cancer is melanoma, bladder cancer, gastric cancer or renal cell carcinoma.

12. The method of any one of the preceding claims, wherein the gene expression ratio computed in step c) indicates that the cancer patient is predicted to respond to anti-cancer immunotherapy, and the method further comprises selecting the cancer patient for anti-cancer immunotherapy.

13. The method of any one of the preceding claims, wherein said anti-cancer immunotherapy comprises immune checkpoint blockade therapy alone or in combination with VEGF inhibition therapy.

14. The method of claim 13, wherein said immune checkpoint blockade therapy comprises programmed death-1 (PD-1) blockade, programmed death-ligand 1 (PD-L1) blockade and/or cytotoxic T-lymphocyte-associated protein 4 (CTLA-4) blockade.

15. The method of claim 1410, wherein said immune checkpoint blockade therapy comprises treatment with Nivolumab, Pembrolizumab, Atezolizumab and/or Ipilimumab.

16. A method of stratifying a plurality of cancer patients according to their method predicted response to anti-cancer immunotherapy, the method comprising carrying out the method of any one of the preceding claims on each of said plurality of cancer patients.

17. A computer-implemented method for predicting the treatment response to anti-cancer immunotherapy of a mammalian cancer patient, the method comprising:
a) providing gene expression data comprising expression levels of at least 2, 3, 4, 5, 6, 7, 8, 9 or more (such as all of) the following cancer promoting genes: PTGS2, VEGFA, CCL2, IL8, CXCL2, CXCL1, CSF3, IL6, IL1B and IL1A previously measured in a sample obtained from the tumour of the patient;
b) providing gene expression data comprising expression levels at least 2, 3, 4, 5, 6, 7, 8, 9, 10, 11, 12, 13, 14 or more (such as all of) the following cancer inhibitory genes: CXCL11, CXCL10, CXCL9, CCL5, TBX21, EOMES, CD8B, CD8A, PRF1, GZMB, GZMA, STAT1, IFNG, IL12B and IL12A in a sample obtained from the tumour of the patient;
c) computing a ratio of the gene expression of said at least 2 cancer promoting genes and the gene expression of said at least 2 cancer inhibitory genes;
d) comparing the computed ratio from step c) with a reference median gene expression ratio derived from a sample cohort of cancer patients having the same type of cancer as said cancer patient; and
e) making a prediction of the treatment response and/or prognosis of the cancer patient based on the comparison made in step d).

18. The method of claim 17, wherein said ratio is of the gene expression of all said cancer promoting genes PTGS2, VEGFA, CCL2, IL8, CXCL2, CXCL1, CSF3, IL6, IL1B and IL1A and all of said cancer inhibitory genes CXCL11, CXCL10, CXCL9, CCL5, TBX21, EOMES, CD8B, CD8A, PRF1, GZMB, GZMA, STAT1, IFNG, IL12B and IL12A.

19. The method of claim 17 or claim 1814, wherein said ratio is calculated according to the formula:
$$COX - 2 ratio = \frac{\frac{1}{{n}_{p}} {\sum }_{i = 1}^{{n}_{p}} {G}_{i}^{pos} ( e )}{\frac{1}{{n}_{n}} {\sum }_{i = 1}^{{n}_{n}} {G}_{i}^{neg} ( e )}$$
wherein nₚ is the number of said cancer promoting genes and nₙ is the number of said cancer inhibitory genes, Gᵢ^{pos} and Gᵢ^{neg} are the positive and negative correlated genes, respectively, within an (i) interval of unitary values, (e) represents the gene expression values, expressed as log 2 counts per million (CPM).

20. The method of any one of claims 17 to 19, wherein the expression level of each of said genes is a normalised gene expression level.

21. The method of claim 17 or claim 18, wherein said ratio is calculated by:
computing the mean gene expression Z-score for said at least 2 cancer promoting genes and the mean gene expression Z-score for said at least 2 cancer inhibitory genes, wherein said z-score is calculated according to the formula
$$z = \frac{x - μ}{σ}$$
wherein z is the gene expression z-score of a given gene, x is the gene expression of the given gene, μ is the mean expression of the given gene in a training set comprising a plurality of cancer subjects and σ is the standard deviation of the gene expression of the given gene in the training set; and
subtracting the Z-score for said at least 2 cancer inhibitory genes from the Z-score for said at least 2 cancer promoting genes.

22. The method of claim 17 or claim 18, wherein said ratio is calculated by:
computing the median gene expression value for each of said at least two cancer promoting genes and said at least two cancer inhibitory genes across a training set comprising a plurality of cancer subjects,
applying, for each of said genes, a value of +1 where the expression value of said cancer patient is greater than the median of that gene over the training set,
summing the cancer inhibitory gene scores and summing the cancer promoting gene scores, and
subtracting the summed cancer inhibitory gene score from the summed cancer promoting gene score, optionally after normalising by gene length.

23. The method of claim 17 or claim 18, wherein said ratio is calculated by:
computing the mean gene expression Z-score for said at least 2 cancer promoting genes and the mean gene expression Z-score for said at least 2 cancer inhibitory genes wherein said z-score is calculated according to the formula
$$z = \frac{x - μ}{σ}$$
wherein z is the gene expression z-score of a given gene, x is the gene expression of the given gene, P is the mean expression of the given gene in a training set comprising a plurality of cancer subjects and σ is the standard deviation of the gene expression of the given gene in the training set;
applying, for each of said genes, a value of +1 where the z-score is greater than 0.1, a value of −1 where the z-score is less than −0.1, and a value of 0 where the z-score is between 0.1 and −0.1;
summing the cancer inhibitory gene applied values and summing the cancer promoting gene applied values, and
subtracting the summed cancer inhibitory gene applied values from the summed cancer promoting gene applied values.

24. The method of claim 17 or claim 18, wherein said ratio is calculated by:
computing the mean gene expression Z-score for said at least 2 cancer promoting genes and the mean gene expression Z-score for said at least 2 cancer inhibitory genes wherein said z-score is calculated according to the formula
$$z = \frac{x - μ}{σ}$$
wherein z is the gene expression z-score of a given gene, x is the gene expression of the given gene, μ is the mean expression of the given gene in a training set comprising a plurality of cancer subjects and σ is the standard deviation of the gene expression of the given gene in the training set;
applying, for each of said genes, a value of +1 where the z-score is greater than 0.3, a value of −1 where the z-score is less than −0.3, and a value of 0 where the z-score is between 0.3 and −0.3;
summing the cancer inhibitory gene applied values and summing the cancer promoting gene applied values, and
subtracting the summed cancer inhibitory gene applied values from the summed cancer promoting gene applied values.

25. A method of treatment of a cancer in a mammalian patient, comprising:
(a) carrying out the method of any or of claims 1 to 15;
(b) determining that the gene expression ratio computed in step c) indicates that the cancer patient is predicted to respond to anti-cancer immunotherapy; and
(c) administering immune checkpoint blockade therapy to the patient in need thereof.

26. The method of claim 25, wherein said immune checkpoint blockade therapy comprises programmed death-1 (PD-1) blockade, programmed death-ligand 1 (PD-L1) blockade and/or cytotoxic T-lymphocyte-associated protein 4 (CTLA-4) blockade.

27. The method of claim 26, wherein said immune checkpoint blockade therapy comprises treatment with a therapeutically effective amount of Nivolumab, Pembrolizumab, Atezolizumab and/or Ipilimumab.

28. The method of any one of claims 25 to 27, wherein said immune checkpoint blockade therapy is combined with anti-VEGF therapy.
